(19) United States
(12) Patent Application Publication
(10) Pub. No.: US 2018/0153826 A1
(43) Pub. Date: Jun. 7, 2018

(54) MATERIALS AND METHODS FOR THE TREATMENT OF CYSTIC FIBROSIS

(71) Applicant: UNIVERSITY OF FLORIDA RESEARCH FOUNDATION, INC., GAINESVILLE, FL (US)

(72) Inventor: SAM XIANJUN CHENG, GAINESVILLE, FL (US)

(21) Appl. No.: 15/571,738

(22) PCT Filed: May 4, 2016
(86) PCT No.: PCT/US2016/030776

§ 371 Date: Nov. 3, 2017

Related U.S. Application Data
(60) Provisional application No. 62/156,606, filed on May 4, 2015.

(51) Int. Cl. A61K 31/137; A61K 31/198; A61K 31/405; A61K 31/4172; A61K 31/4045; A61K 45/06; A61K 9/00; A61P 11/12

(52) U.S. Cl. CPC A61K 31/137; A61K 31/198; A61K 31/405; A61P 11/12; A61K 31/4045; A61K 45/06; A61K 9/0078; A61K 31/4172

(57) ABSTRACT

In preferred embodiments, the invention pertains to the treatment of Cystic Fibrosis (CF) using an agonistic of CaSR. In a specific embodiment, the subject invention provides a method of treating CF in a subject by administering to the subject a composition comprising a calcimimetic. The calcimimetic can be administered alone or in combination with a CaSR orthosteric agonist and/or an agent capable of stimulating colonic HCO₃₋ secretion in CFTR-independent manner. The composition can be administered to the subject via inhalation. Accordingly, the invention further pertains to compositions comprising a calcimimetic and optionally, further comprising a CaSR orthosteric agonist and/or an agent capable of stimulating colonic HCO₃₋ secretion in CFTR-independent manner, in the form suitable for administration to the subject via inhalation. Accordingly, devices for administering the compositions of the current invention via inhalation are also provided.

### CROSS-REFERENCE TO A RELATED APPLICATION

This application claims the benefit of U.S. Provisional Patent Application Ser. No. 62/156,606, filed May 4, 2015, which is incorporated herein by reference in its entirety.

This invention was made with government support under HD079674 (SXC) awarded by National Institutes of Health. The government has certain rights in the invention.

### BACKGROUND OF THE INVENTION

Defective bicarbonate (HCO₃⁻) secretion by transport epithelia is a hallmark of pathophysiology in patients with cystic fibrosis (CF). In CF, defective bicarbonate secretion results in defective mucus secretion, leading to respiratory airway epithelial malfunction and infections, bowel obstructions, pancreatic insufficiency, liver disease and infertility. Despite worldwide efforts in searching for methods to correct this defect, so far an efficacious method is not found. Current CF therapies include CFTR gene therapy, CFTR chaperones therapy, CFTR stimulators and other approaches are designed to rescue CFTR function.

### BRIEF SUMMARY

The subject invention provides methods for treating cystic fibrosis (CF) in a subject by administering to the subject a composition comprising a CaSR agonist. Specifically exemplified herein is the use of a CaSR type II agonist together with a type I agonist and/or a co-factor. In one embodiment the agonist is a calcimimetic in combination with a CaSR orthosteric (type I) agonist and/or an agent capable of stimulating colonic HCO₃⁻ secretion in a CFTR-independent manner.

In addition to treating CF, the compositions and methods of the subject invention can be used to generally promote and/or improve mucosal health.

In one embodiment, the method comprises treating a subject by administering a composition of the invention to the subject via inhalation. In another embodiment, the composition is administered via enema. In one embodiment the patient has been diagnosed with CF.

Accordingly, certain embodiments of the invention provide compositions for treating CF in a form suitable for administration to the subject via inhalation.

Devices for administration of the compositions of the invention to a subject via inhalation are also provided.

### BRIEF DESCRIPTION OF THE DRAWINGS

The file of this patent application contains at least one drawing executed in color. Copies of this patent with color drawings(s) will be provided by the Patent and Trademark Office upon request and payment of the necessary fee.

FIGS. 1A-1B. Anion transport defects in CF.

FIGS. 2A-2B. CaSR can uncouple HCO₃⁻ secretion mediated by CFTR from non CFTR transporters and differentially regulate them.

FIG. 3. Experimental approach 1: The Ussing chamber pH-stat set up used to monitor and measure HCO₃⁻ secretion by colonic mucosa.

FIG. 4. Experimental Approach 2: Measurement of HCO₃⁻ secretion by colonic mucosa in the presence of apical NPPB/Glibenclamide.

FIGS. 5A-5F. Activation of CaSR by R568 inhibits CFTR-mediated HCO₃⁻ secretion (J_{HCO3}) and I_{SC} but stimulates non CFTR-mediated HCO₃⁻ secretion (J_{HCO3}) and I_{sc}. Physiological approach assayed with, versus without, lumen Cl⁻.

FIGS. 6A-6F. Activation of CaSR by R568 inhibits CFTR-mediated HCO₃⁻ secretion (J_{HCO3}) and I_{SC} but stimulates CFTR-independent HCO₃⁻ secretion (J_{HCO3}) and I_{sc}. Pharmacological approach assayed with, and without, CFTR transporter inhibitors or AE inhibitor DIDS.

FIGS. 7A-7C. Effects of extracellular Ca²⁺ and CaSR agonist R568 on basal HCO₃⁻ secretion. A-B: Representatives of 3 recordings of luminal pH responses of distal colonic mucosa to absence and presence of a serosa-to-lumen directed HCO₃⁻ gradient and carbachol (CCH, 100 μM, serosa) (A), and normal vs. low [Ca²⁺]ₒ. (B). The presence or absence of HCO₃⁻ in the lumen or serosa was shown as indicated. C: Serosal-to-mucosal J_{HCO3}⁻ responses to R568 (10 μM, serosa). The basal tissue resistance (Ωcm²) and I_{sc} (μEq/hr/cm²) were 69±7 and 1.44±0.32 at 0.5 mM [Ca²⁺]ₒ vs. 72±6 and 1.10±0.10 at 1.2 mM [Ca²⁺]ₒ (p>0.05). Data are means±SEM of 5 experiments. *p<0.05 vs. control (with no R568).

FIGS. 8A-8B. Effect of CaSR agonist R568 on acid-induced HCO₃⁻ secretion. A: Representatives of pH recovery responses in the presence vs. absence of R568 (10 μM, serosa). The addition of R568 is shown as indicated. B: Summary of ApH recovery rates (stimulated peak values above basal levels before additions of R568 or vehicle). Basal levels of acid-induced pH recovery were 0.015±0.001 pH unit/min/cm². The tissue resistance (Ωcm²) and I_{sc} (μEq/hr/cm²) at the end of 60 min experiments were 50±12 and 1.74±0.20 without challenge and 46±10 and 1.97±0.23 with acid challenges (p>0.05). Data are means±SEM of 5 experiments. *p<0.05 vs. control (with no R568).

FIGS. 9A-9D. Effect of CaSR agonist R568 on secretagogue-induced HCO₃⁻ secretion and HCO₃⁻ current. Shown are J_{HCO3} (A) and I_{sc} (C) responses to forskolin (FSK, 500 nM, serosa)±R568 (10 μM, serosa), assayed in lumen Cl⁻-containing Ringer solution. The changes induced by FSK in the absence vs. presence of R568 are summarized in B (ΔJ_{HCO3}^{FSK}) and D (ΔI_{sc}^{FSK}). The basal tissue resistance and I_{sc} under these conditions were: 68±4 Ωcm² and 1.51±0.22 μEq/hr/cm². Data are means±SEM of 8 experiments. **p<0.01 vs. control (with no R568); ## p<0.01 vs. FSK.

FIGS. 10A-10B. Activation of CaSR stimulates Cl⁻-dependent HCO₃⁻ secretion. A: J_{HCO3} responses to the presence vs. absence of lumen Cl⁻. B: the ΔJ_{HCO3}^{Cl} (i.e., Cl⁻-dependent HCO₃⁻ secretion) responses to the presence vs. absence of R568 (10 μM, serosa). The basal tissue resistance (Ωcm²) and I_{sc} (μEq/hr/cm²) were 68±11 and 1.44±0.32 in the presence vs. 95±15 and 1.01±0.08 in the absence of lumen Cl⁻ (p>0.05). Data are means±SEM of 3-5 experiments. *p<0.05 and **p<0.01 vs. control (1^{st} bar).

FIGS. 11A-11D. Activation of CaSR stimulates SCFA-dependent HCO₃⁻ secretion. Two equally longitudinally divided pieces of mucosa from same colon were mounted into 2 Ussing chambers. One piece was treated first in the presence then absence of lumen SCFA whereas the other piece was treated first in the presence then absence of serosa HCO₃⁻. Shown are representative recordings (A & B) and quantitative summary (C) of these J_{HCO3} responses. The ΔJ_{HCO3}^{sCFA} (i.e., SCFA-dependent HCO₃⁻ secretion) was calculated and its responses to the presence vs. absence of R568 (10 μM, serosa) are shown in D. The basal tissue resistance (Ωcm²) and I_{sc} (μEq/hr/cm²) were 126±26 and 0.71±0.17 in the presence vs. 95±5 and 1.01±0.08 in the absence of lumen 25 mM isobytyrate (p>0.05). Data are means±SEM of 3-5 experiments. *p<0.05 and **p<0.01 vs. control (1^{st} bar).

FIGS. 12A-12D. Activation of CaSR inhibits cAMP-dependent HCO₃⁻ secretion and HCO₃⁻ current. Shown are J_{HCO3} (A) and 1: (C) responses to forskolin (FSK, 500 nM, serosa) R568 (10 μM, serosa), assayed in lumen Cl⁻-free Ringer solution. The changes induced by FSK in the absence vs. presence of R568 are summarized in B (ΔJ_{HCO3}^{FSK}) and D (ΔI_{SC}^{FSK}) The basal tissue resistance and I_{sc} under these conditions were 103±3 Ωcm² and 0.87±0.05 μEq/hr/cm². Data are means±SEM of 4-7 experiments. **p<0.01 vs. control (1^{st} bar); ## p<0.01 vs. FSK alone (2^{nd} bar).

FIGS. 13A-13F. R568 fails to stimulate Cl⁻- and SCFA-dependent and inhibit cAMP-dependent HCO₃⁻ secretion in colons of CaSR null mice. Colonic mucosa from wild type (CaSR^{+/+}) (A-C) and knockout (CaSR^{-/-}) mice (D-F) were isolated and treated, and activities of the three HCO₃⁻ transporters assayed as in rats. Data are means±SEM of 4-6 experiments. *p<0.05 vs. control (1^{st} bar).

FIG. 14. Cellular model of CaSR regulation of HCO₃⁻ secretion in colonocytes of rat distal colon. Upper panel: R568 acting via CaSR causes enhancement of HCO₃⁻ secretion in surface epithelial cells by stimulation of luminal Cl⁻-dependent HCO₃⁻ secretion via apical Cl⁻/HCO₃⁻ exchange and stimulation of luminal SCFA-dependent HCO₃⁻ secretion mediated by apical SCFA/HCO₃⁻ exchange. Lower panel: CaSR reduces HCO₃⁻ secretion in the crypt epithelial cells through inhibition of cAMP-dependent HCO₃⁻ secretory process that may involve a NPPB/glibenclamide-sensitive apical anion channel such as CFTR and/or a basolateral HCO₃⁻ entry mechanism(s), +, stimulation; −, inhibition.

FIG. 15. Cation transport defect in CF.

FIG. 16. CaSR inhibits ENaC and R568 corrects CF-associated defect in cation transport.

FIG. 17. The inhibitory effect of R568 on ENaC was examined electrophysiologically by measuring amiloride-sensitive short-circuit current responses in the presence or absence of R568. Weanling infant distal colonic mucosa, which expresses highest activity of ENaC, is employed and mounted in Ussing chamber.

FIGS. 18A-18C. Representatives (A-B) and summary (C) of 10 μM amiloride effects on basal I_{sc} in 2-3 week old Sprague-Dawley rat proximal (A) and distal (B) colon.

FIGS. 19A-19C. Representative (A-B) and summarized (C) rate changes in I_{sc} by 10 μM R568 in 2-3 week old Sprague-Dawley rat distal colon. Here, the activity of ENaC is measured as amiloride-sensitive bumetanide-insensitive I_{sc}. The results indicate that activation of CaSR by R568 inhibited the activity of ENaC.

FIGS. 20A-20C. Representative (A-B) and summarized (C) changes in I_{sc} in proximal vs. distal colons of 3 week old Sprague-Dawley rats fed with normal (1% calcium) vs. high calcium (2.5%) diet. The activity of ENaC was measured as amiloride-sensitive bumetanide-insensitive I_{sc}. Activation of CaSR by dietary calcium suppressed the activity of ENaC.

FIG. 21. Activation of CaSR inhibits activity and function of ENaC.

### DETAILED DISCLOSURE

The invention is based, in part, on the identification of the role of CaSR in HCO₃⁻ secretion, including the role of CaSR in basal, acid- and secretagogue-induced HCO₃⁻ secretions. The role of CaSR in normal physiology as well as during pathophysiology, such as, diarrhea and CF has been identified. Further, the effects of CaSR agonists, for example, R-568, on Cl⁻/HCO₃⁻ and SCFA/HCO₃⁻ exchanges (which are electroneutral and can be measured by pH stat) and electrogenic HCO₃⁻ movement (which can be measured by I_{sc} and pH stat) are provided. In accordance with the subject invention CaSR agonists can be used to improve mucosal physiology through differentially regulating HCO₃⁻ secretion.

CF is caused by mutations in CFTR that cause defects in transepithelial Cl⁻ transport resulting in inability to maintain luminal hydration and defects in transepithelial HCO₃⁻ transport resulting in inability to secrete alkaline fluid. This leads to mucus plugging and destruction of affected organs in CF. Accordingly, CF affects the function of multiple organs.

HCO₃⁻ is biological buffer that maintains acid-base balance, thereby preventing metabolic and respiratory acidosis. HCO₃⁻ also buffers the pH of mucosal layers that line all epithelia, protecting them from injury. Being a chaotropic ion, HCO₃⁻ is essential for solubilization of ions and macromolecules such as mucins and digestive enzymes in secreted fluids. HCO₃⁻ is of particular importance in CF because pH and HCO₃⁻ affect mucin viscosity and binding of bacteria to mucins.

The CF phenotype varies from very severe with pancreatic insufficiency (PI) to very mild with pancreatic sufficiency (PS). CFTR mutations causing no defect in HCO₃⁻ transport result in mild CF phenotype with PS; whereas, CFTR mutations causing defects in HCO₃⁻ transport produce severe CF phenotype with PI. Accordingly, in accordance with the subject invention, stimulating HCO₃⁻ transport in CF is used in the prevention and/or treatment of CF.

The mechanism of HCO₃⁻ secretion by CFTR-expressing epithelia is not well understood. HCO₃⁻ is secreted by two pathways mediated by CFTR (CFTR dependent manner) and non CFTR transporters (CFTR-independent manner). These two pathways are coupled together and diseases that turn off one pathway typically turn off the other.

In accordance with the subject invention it has been found that CaSR “uncouples” HCO₃⁻ secretion mediated by CFTR-dependent from CFTR-independent mechanisms and differentially regulates them. In the absence of a CaSR agonist, for example, R568, CFTR couples with electrogenic PAT1 and electroneutral AE. As a consequence, stimulation of CFTR stimulates PAT1 but inhibits AE1 (FIG. 2A). In the presence of a CaSR agonist, for example, R568, the CFTR interactions with PAT1 and AE1 are “uncoupled”. As a consequence, CFTR and PAT1 are inhibited but AE1 is stimulated (FIG. 2B).

In accordance with the subject invention, activation of CaSR by a CaSR agonist, for example, R568, inhibits CFTR-dependent HCO₃⁻ secretion but stimulates CFTR-independent HCO₃⁻ secretion.

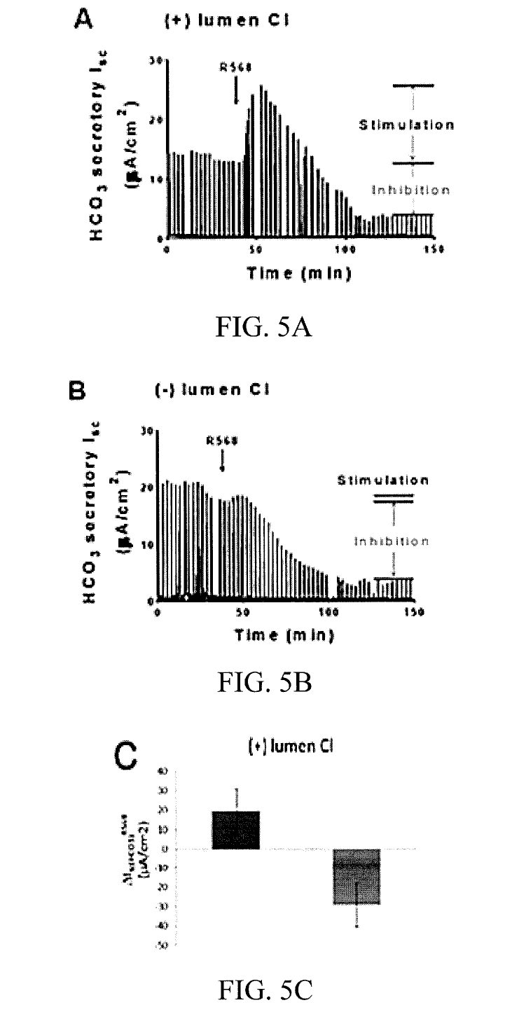

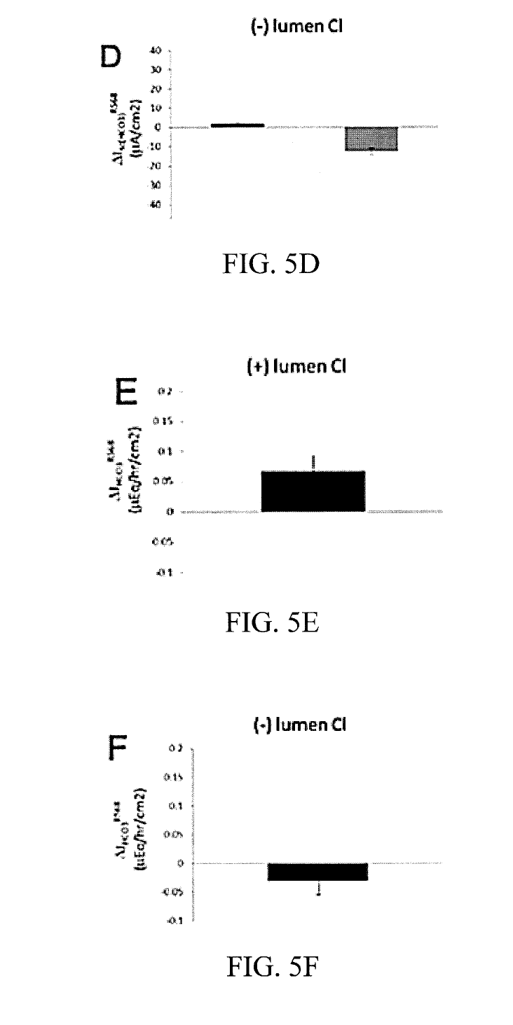

In the presence of lumen Cl⁻, R568 was found to induce a two-phase response (FIG. 5A): an immediate phase I I_{sc} stimulatory response followed by a sustained phase II I_{sc} inhibitory response. Phase I (red column) and II (blue column) responses are quantified and shown in FIG. 5C. Despite different effects on HCO₃⁻ secretory currents, R568 induced a net stimulation of HCO₃⁻ secretion, as revealed in pH stat measurements (FIG. 5E).

In the absence of lumen Cl⁻, the phase I stimulation induced by R568 was abolished (FIGS. 5B and 5D) suggesting that the stimulation by R568 is mediated by Cl⁻/HCO₃⁻ anion exchange; however, the phase II inhibition remained unchanged or slightly reduced (FIGS. 5B and 5D), consistent with inhibition of a Cl⁻-independent mechanism (e.g., CFTR). Under these conditions, R568 induced a net inhibition of HCO₃⁻ secretion (FIG. 5F).

The results suggest that R568 inhibits CFTR-dependent HCO₃⁻ secretion but stimulates CFTR-independent HCO₃⁻ transport (e.g., Cl⁻-dependent HCO₃⁻ secretion). The Cl⁻/HCO₃⁻ exchange stimulated by R568 is electrogenic with stoichiometry consistent with Solute carrier family 26 member 6 (Slc26A6), an AE that mediates 1Cl⁻/2HCO₃⁻ exchange and generates a lumen negative potential difference (PD) and positive I_{sc}. R568 stimulation of Cl⁻/HCO₃⁻ exchange is associated with more inhibition of CFTR activity; however, the net effect of R568 in the presence of lumen Cl⁻ is stimulatory in HCO₃⁻ secretion (FIG. 5E).

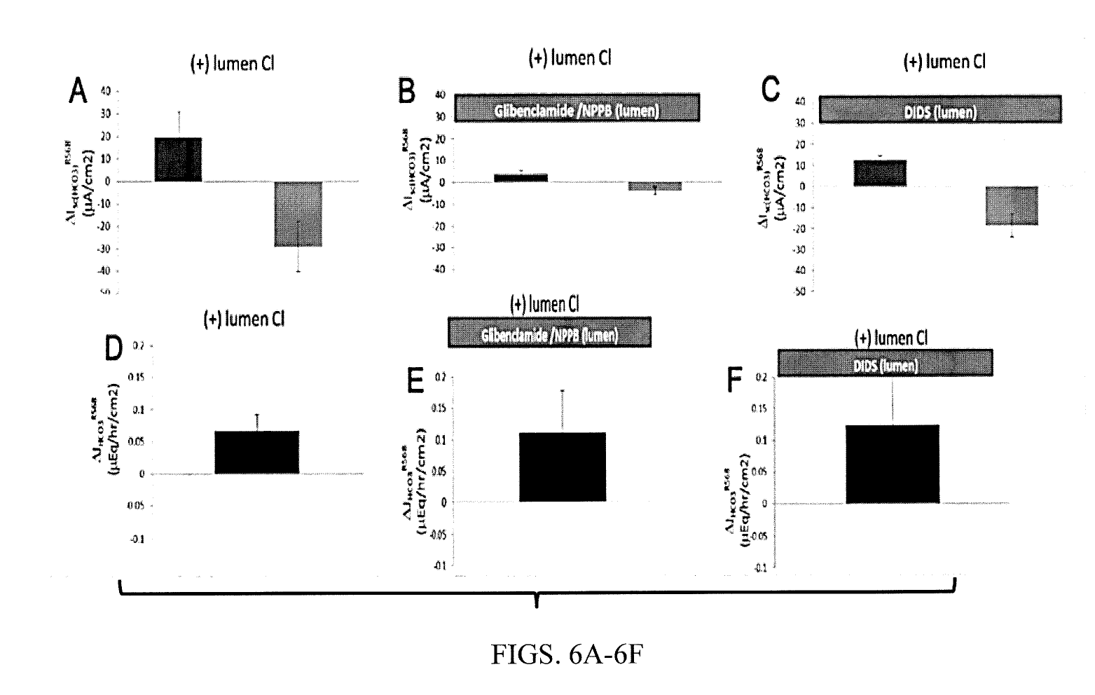

Inhibition of CFTR by NPPB/Glibenclamide diminished, not only I_{sc-CFTR} (blue columns in FIG. 6B vs. 6A) but surprisingly also I_{sc-AE} (red columns in FIG. 6B vs. 6A) while the actual HCO₃⁻ secretion remained unchanged or slightly stimulated (FIG. 6E with 6D). These data suggest that the function of AE requires an active CFTR and that an additional HCO₃⁻ secretory mechanism that is normally inactivated by CFTR might now be reactivated upon CaSR stimulation by R568.

Similarly, 4,4′-diisothiocyano-2,2′-stilbenedisulfonic acid (DIDS) partially inhibited not only the AE-associated current (red columns of FIG. 6C vs. 6A) but also inhibited the CFTR activity (blue columns of FIG. 6C vs. 6A). These data suggest that the function of CFTR also requires an active AE. Again, under these conditions the HCO₃⁻ secretion rate was stimulated or did not change (FIG. 6F with 6D).

These results indicate that CFTR controls two independent HCO₃⁻ secretory mechanisms in the colon: it stimulates the electrogenic AE (e.g., PAT1) while it simultaneously inhibits the other mechanism (possibly not a channel but an electroneutral AE (e.g., AE1)). CaSR may function as an “uncoupler”, separating the function of CFTR from non CFTR transporters and differentially regulating them.

In accordance with the subject invention, agents and methods that uncouple and separately turn on/off CFTR-dependent and CFTR-independent HCO₃⁻ secretion provide novel therapies for treating CF. Accordingly, an embodiment of the invention provides a method of treating CF in a subject. The method comprises administering to the subject, a CaSR agonist. The CaSR agonist can be an orthosteric agonist (type I agonist), which is capable of activating the CaSR on its own, or an allosteric agonist, which binds to allosteric sites on CaSR and requires the binding of an orthosteric agonist to the receptor to produce the agonistic effects.

In one embodiment, the subject invention provides a CaSR agonist, for example, R568, as an agent for the treatment of CF and/or to promote mucosal health, including preventing and/or treating diarrheal illnesses.

Ca²⁺ is the primary orthosteric agonist of the CaSR. Other orthosteric agonists include divalent and trivalent cations, including Mg²⁺, Al³⁺, Sr²⁺, Mn²⁺, Ni²⁺, Gd³⁺, and Ba²⁺; aminoglycoside antibiotics (e.g. neomycin, gentamycin, tobramycin) and polyamines (e.g. spermine, spermidine, putrescine), all of which are positively charged. In general, CaSR agonists with a high positive charge density tend to have higher potency.

Allosteric agonists of CaSR are also called calcimimetics. Allosteric agonists of CaSR include, but are not limited to, aromatic L-amino acids (e.g., L-phenylalanine, L-tryptophan, L-tyrosine, L-histidine) and small molecule calcimimetics. Non-limiting examples of small molecule calcimimetics include R568 (2-Chloro-N-[(1R)-1-(3-methoxyphenyl)ethyl]-benzenepropanamine hydrochloride), R467 ((R)—N-(3-phenylpropyl)-α-methyl-3-methoxybenzylamine hydrochloride), Cinacalcet ((R)—N-[(1-naphthyl)ethyl]-3-[3-(trifluoromethyl)phenyl]propan-1-amine), Calindol ((R)-2-[[[1-(1-Naphthyl)ethyl]amino]methyl]-1H-indole Hydrochloride). Additional examples of small molecule calcimimetics are known to a person of ordinary skill in the art and such embodiments are within the purview of the claimed invention.

Examples of calcimimetics can be found in U.S. Pat. Nos. 8,791,147; 8,609,655; 8,486,381; 8,349,831; and 8,334,317, all of which are incorporated herein by reference, in their entireties.

In one embodiment of the subject invention, the method of treating CF in a subject comprises administering to the subject a composition comprising a calcimimetic. The calcimimetic can be selected from, for example, L-phenylalanine, L-tryptophan, L-tyrosine, L-histidine, R568, R467, Cinacalcet, and Calindol. Additional examples of a calcimimetic are well known to a person of ordinary skill in the art and such embodiments are within the purview of the current invention.

In a further embodiment of the subject invention, the method of treating CF in a subject comprises administering to the subject a composition comprising a calcimimetic and a CaSR orthosteric agonist. For example, CF can be treated by administering a composition comprising R568 and Ca²⁺. Any combination of a calcimimetic and a CaSR orthosteric agonist known to a person of ordinary skill in the art can be used to treat CF in a subject. For example, a person of ordinary skill in the art can combine any of the aforementioned calcimimetics or another calcimimetic known in the art with any of the aforementioned CaSR orthosteric agonists or another CaSR orthosteric agonist known in the art to produce a composition for treatment of CF.

In another embodiment, the method of treating CF in a subject comprises administering to the subject a composition comprising a calcimimetic and an agent capable of stimulating colonic HCO₃ secretion in a CFTR-independent manner. Non-limiting examples of agents capable of stimulating colonic HCO₃⁻ secretion in a CFTR-independent manner include Cl⁻ and SCFA. Additional examples of agents capable of stimulating colonic HCO₃⁻ secretion in a CFTR-independent manner are well known to a person of ordinary skill in the art and such embodiments are within the purview of the invention.

In one embodiment, the method of treating CF in a subject comprises administering to the subject a composition comprising a calcimimetic and an agent capable of stimulating colonic HCO₃⁻ secretion in a CFTR-independent manner. For example, CF can be treated by administering a composition comprising R568 and SCFA. Any combination of a calcimimetic and an agent capable of stimulating colonic HCO₃⁻ secretion in a CFTR-independent manner known to a person of ordinary skill in the art can be used according to the subject invention to treat CF in a subject. For example, a person of ordinary skill in the art can combine any of the aforementioned calcimimetics or another calcimimetic known in the art with any of the aforementioned agents capable of stimulating colonic HCO₃⁻ secretion in a CFTR-independent manner or another agent capable of stimulating colonic HCO₃⁻ secretion in a CFTR-independent manner known in the art to produce a composition for treatment of CF.

In a further embodiment of the invention, the composition comprising a calcimimetic and an agent capable of stimulating colonic HCO₃⁻ secretion in a CFTR-independent manner further comprises a CaSR orthosteric agonist. Accordingly, in one embodiment, the method of treating CF comprises administering, to a subject in need thereof, a composition comprising a calcimimetic, an agent capable of stimulating colonic HCO₃⁻ secretion in a CFTR-independent manner and a CaSR orthosteric agonist. Accordingly, one specific embodiment of the invention provides treating CF in a subject by administering to the subject a composition comprising R568, SCFA and Ca²⁺.

Table 1 provides examples of calcimimetics, agents capable of stimulating colonic HCO₃⁻ secretion in a CFTR-independent manner, and CaSR orthosteric agonists. Any combination of a calcimimetic, with either or both of an agent capable of stimulating colonic HCO₃⁻ secretion in a CFTR-independent manner and a CaSR orthosteric agonist is within the purview of the invention.

TABLE 1
Examples of calcimimetics, agents capable of stimulating colonic
HCO₃⁻ secretion in a CFTR-independent manner and CaSR orthosteric agonists.

| | Agent capable of stimulating | |
|---|---|---|
| | colonic HCO₃⁻ secretion in a | |
| Calcimimetic | CFTR-independent manner | CaSR orthosteric agonist |
| R568, R467, Calindol, | Cl⁻, SCFA | Ca²⁺ₒ, Mg²⁺, Al³⁺, Sr²⁺, Mn²⁺, |
| Cinacalcet, L-phenylalanine, | | Ni²⁺, Gd³⁺, Ba²⁺, neomycin, |
| L-tryptophan, L-tyrosine, L- | | gentamycin, tobramycin, |
| histidine | | spermine, spermidine, |
| | | putrescine |

Additional examples of calcimimetics, agents capable of stimulating colonic HCO₃⁻ secretion in a CFTR-independent manner, and CaSR orthosteric agonists are well known to a person of ordinary skill in the art and combinations of such compounds are also within the purview of the claimed invention.

### Pharmaceutical Compositions

Certain embodiments of the invention provide pharmaceutical compositions comprising one or more of the compounds described herein and a pharmaceutically acceptable carrier and/or excipient. Pharmaceutical compositions, as disclosed herein, can be formulated in accordance with standard pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

Compositions for parenteral administration are generally physiologically compatible sterile solutions or suspensions which can optionally be prepared immediately before use from solid or lyophilized form. Adjuvants such as a local anesthetic, preservative and buffering agents can be dissolved in the vehicle and a surfactant or wetting agent can be included in the composition to facilitate uniform distribution of the active ingredient.

For oral administration, the composition can be formulated into conventional oral dosage forms such as tablets, capsules, powders, granules and liquid preparations such as syrups, elixirs, and concentrated drops. Non toxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials are also necessary. For example, starch, gelatin, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

For transdermal administration, the composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compound can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

In a particular embodiment, the method of treating CF comprises administering to a subject in need thereof, a composition comprising a calcimimetic and optionally further comprising an agent capable of stimulating colonic HCO₃⁻ secretion in a CFTR-independent manner and/or CaSR orthosteric agonist via inhalation.

Compositions of the invention appropriate for administration via inhalation can be a solution, suspension, or powder. These formulations are typically administered via an aerosol or a dry powder inhaler. Aerosol is a colloidal suspension of particles dispersed in air or gas. In aerosols, liquid or suspension droplets are the internal phase and a gas is the external phase.

In one embodiment, an aerosol delivery device is used to administer the compositions of the invention. An aerosol delivery device is used to produce aerosols, for example, for delivery to a subject via inhalation. Metered dose inhalers (MDI) are aerosol delivery devices that deliver a fixed dose in a spray with each actuation of the device.

In certain embodiments, atomizers, nebulizers, or vaporizers are used as aerosol delivery devices. Additional examples of aerosol delivery devices are well known to a person of ordinary skill in the art and such embodiments are within the purview of the current invention.

Atomizers break up a liquid into an aerosol. Typically, an atomizer comprises a squeeze bulb which is used to blow air into the device causing the drug solution to rise in a small dip tube and vaporizing in the air stream. The air stream is directed into a baffle or bead which breaks the droplets in to even smaller droplets as they collide with the device. The mixture of air and liquid then exits the atomizer in the form of an aerosol.

A nebulizer contains an atomizing unit within a chamber. When the rubber bulb is depressed, the medication solutions is drawn up a dip tube and aerosolized by the passing air stream. Baffles or beads may also be present in the chamber. The fine droplets exit the nebulizer. The larger droplets collect on the chamber and fall back into the reservoir where they can be used again.

Vaporizers produce a fine mist of steam. Volatile medication is added to the water in the vaporizer or to a special medication cup present in some models. The medication volatilizes and is inhaled by a patient as he/she breathes.

In one embodiment, the composition is a dry powder and the inhalers contain the dry powders in cartridges or disks. When a patient administers a dose, the device is first activated by some mechanical motion and the dry powder becomes ready for inspiration. The patient then inhales through the device mouthpiece and the powder is drawn into the pulmonary tract along with the inspired air. These devices have overcome a major problem of inhalation therapy, synchronizing deep inspiration with the administration of the drug. Some of the commercially available devices are Diskhaler®, Turbuhaler®, Diskus®, and Rotahaler®.

In a further embodiment, a powdered composition is administered with insufflators or puffers. Squeezing the rubber bulb of an insufflator causes turbulence within the powder reservoir which forces some of the powder into the air stream and out of the device. A puffer is a plastic accordion-shaped container with a spout on one end. The powder is placed inside the container and the puffer is actuated by squeezing the device. A portion of the powder is ejected from the spout.

Additional devices appropriate for administration of the composition of the claimed invention via inhalation are well known to a person of ordinary skill in the art and such embodiments are within the purview of the invention. For example, Advanced Drug Delivery Reviews (2014), Volume 75, Pages 1-148 contains several articles directed to “Iimproving the efficacy of inhaled drugs for severe lung diseases: emerging pulmonary delivery strategies.” The contents of these articles are herein incorporated by reference in their entirety, particularly, Angelo et al., “Improving the efficacy of inhaled drugs in CF: Challenges and emerging drug delivery strategies.” Certain devices and methods for delivery of therapeutic substances via inhalation are also described “A Guide to Aerosol Delivery Devices for Respiratory Therapists, 3rd Edition (2013)” published by American Association for Respiratory Care, the contents of which are incorporated herein by reference in their entirety.

### Definitions

Terms such as “treating,” “treatment,” “to treat,” refer to both 1) therapeutic measures that cure, slow down, lessen symptoms of, and/or halt progression of CF, and 2) prophylactic or preventive measures that prevent or slow the development of CF. Thus, those in need of treatment include those already with CF; those prone to having CF; and those in whom the CF is to be prevented. A subject is successfully “treated” according to the methods of the present invention if the patient shows one or more of the following: mucus clearance, absence of infection, good lung function, slower progression of lung disease, reduced inflammation, improved respiratory function, reduced exacerbations, increased mucociliary clearance, loosening and removal of thick, sticky mucus from the lungs, avoidance of blockages in the intestines, prevention of dehydration and reduction in problems with digestive system. Complete absence of any CF symptoms is not required for successful “treatment.”

The term “administering” is defined herein as a means of providing an agent or a composition containing the agent to a subject in a manner that results in the agent being inside the subject's body. Such an administration can be by any route including, without limitation, oral, subcutaneous, intradermal, intravenous, intra-arterial, intratumoral, intraperitoneal, intramuscular, or via inhalation.

The term “subject” or “patient” refers to an animal which is the object of treatment, observation, or experiment. By way of example only, a subject includes, but is not limited to, a mammal, including, but not limited to, a human or a non-human mammal, such as a bovine, equine, canine, ovine, murine or feline. In certain embodiments, the treatment of humans is contemplated by this invention.

The term “effective amount” means the amount of an agent required to treat CF and to produce some desired therapeutic effect. The effective amount of compound(s) used to practice the present invention for prevention or treatment of CF varies depending upon the manner of administration, the age, body weight, and general health of the subject. Ultimately, the attending physician will decide the appropriate amount and dosage regimen.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### Materials and Methods

### Animals and Tissue Preparations

Experiments were performed using non-fasting male Sprague-Dawley rats and C57BL/6 mice. Rats weighting 100-400 g were obtained from Charles River Laboratories. Mice lacking CaSR expression in intestinal epithelial cells (CaSR^{-/-}) and their CaSR^{+/+} littermates were bred and maintained. CaSR^{-/-} mice were generated as previously described. Briefly, CaSR flox/flox mice were bred with transgenic mice expressing Cre Recombinase under the control of the villin 1 promoter and genotyped prior to all experiments after an approximate 10-12 generations. Mice were used at 5-10 weeks of age. Animals were fed and maintained on regular chow (Harlan) with free access to water before sacrifice. Animals were sacrificed with standard CO₂ inhalation followed by cervical dislocation. The colons were isolated, cut open along the mesenteric border into a flat sheet, and flushed with ice-cold Ringer solutions. Mucosa from the distal colon were carefully hand-stripped off of serosal, muscular and submucosal layers as described and a pair of adjacent mucosal segments were incised and mounted into Ussing chambers. In some experiments, stripped mucosa was incised longitudinally into two equal pieces in order to facilitate comparisons between control and treatment. Short-circuit current and transepithelial resistance differences between these two adjacent tissues were less than 15%.

### Ussing Chamber Setup

Stripped mucosal sheets were mounted into Ussing chambers (window area=0.3-0.5 cm², Physiologic Instruments, San Diego, Calif.). The mucosal side of tissue was bathed with an unbuffered HCO₃⁻-free Cl⁻ Ringer solution (see Table 2 for detailed composition) circulated by a gas lift with 100% O₂ while the serosal side was bathed with buffered Cl⁻ Ringer solution (pH 7.4) that contained 25 mM HCO₃⁻ and gassed with 5% CO₂/95% O₂. Each side contained 3-5 ml of solution and the temperature of the solution was adjusted to and maintained at 37° C. by heated water-jacketed reservoirs. Experiments were performed under short-circuit conditions (Voltage-Current Clamp, VCC MC8; Physiologic Instruments, San Diego, Calif.) to maintain the transepithelial potentials at 0 mV, except for brief interruption at 20-second intervals for recording of open-circuit potential (V_{T}, mV).

TABLE 2
Composition of Ringer solutions

| | Serosa | | | Lumen | | | |
|---|---|---|---|---|---|---|---|
| Compo- | Cl⁻/ | HCO₃⁻ | Cl⁻ | Cl⁻/ | HCO₃⁻ | | |
| nent | HCO₃⁻ | free | free | HCO₃⁻ | free | Cl⁻ | SCFA |
| Cl⁻ | 123 | 123 | | 123 | | 123 | |
| HCO₃⁻ | 25 | | 25 | 25 | | | |
| Iso- | | 25 | 122 | | 147 | 25 | 122 |
| thionate | | | | | | | |
| Iso- | | | | | | | 25 |
| butyrate | | | | | | | |
| SO₄⁻ | | | 1 | | 1 | | 1 |

Two types of approaches were used in the measurement of colonic HCO₃⁻ secretion, namely, measurement of lumen pH or pH stat titration of net alkalinization by colonic mucosa. Except for limited experiments that require direct measurements of lumen pH (e.g., Experiment 1 and Experiment 3 below), luminal pH was maintained at 7.4 by the continuous infusion of 1 mM HCl or H₂SO₄ in case of lumen Cl⁻-free conditions under the automatic control of a pH-stat system (Bi-burette TIM 856 pH meter; Radiometer Analytical, Villeurbanne, France). The amount of the acid delivered per unit time per surface area was used to quantitate HCO₃⁻ secretion by the mucosa. Measurements were recorded continuously and mean values for consecutive 5 or 10 min periods were averaged. The rate of HCO₃⁻ secretion (J_{HCO3}) is expressed as μEq/hr/cm². The short-circuit current (I_{sc}) was measured in microamperes (μA) and converted into μEq/hr/cm². Tissue resistance (R, Ω cm²) was calculated from Ohm's law.

Experimental Designs—Seven Experiments for HCO3- Secretion were Conducted.
Lumen Alkalinization Response to [Ca²⁺]ₒ

Two equal pieces of mucosa from each colon that were obtained by longitudinal division along the antimessenteric border were mounted into two Ussing chambers. One piece was bathed with Cl⁻ Ringer solution that contained 1.2 mM [Ca²⁺]ₒ, while the other piece was bathed with Cl⁻ Ringer solution that contained 0.5 mM [Ca²⁺]ₒ. Initially, tissues were bathed, both luminally and serosally, in HCO₃⁻-free solution. After 15 to 30 min, when stabilization was achieved and basal lumen pH recordings performed, HCO₃⁻-free Ringer solution in the serosal side was replaced by 25 mM HCO₃-containing Ringer solution, and changes in lumen pH were monitored and recorded for 15 to 30 min. In some tissues, carbachol was added to the serosal side before the experiment was concluded.

Basal HCO₃⁻ Secretion

Two adjacent sheets of mucosa from each colon were mounted into two Ussing chambers and bathed luminally with HCO₃⁻-free Cl⁻ Ringer solution and serosally with HCO₃⁻-containing Cl⁻ Ringer solution. After 15 to 30 min, when I_{sc} and J_{HCO3} had stabilized, the CaSR agonist, R568 was added to the serosal or mucosal side of tissue, and changes in I_{sc} and J_{HCO3} during the 15-30 min period ensuing after the addition of the agonist were determined.

Acid-Induced HCO₃⁻ Secretion

Colon mucosa from each animal was divided longitudinally into 2 equal pieces and mounted into two Ussing chambers. Acid (hydrochloride) was added to the lumen to lower pH by approximately 0.3-0.4 units and changes in lumen pH monitored. In response to luminal addition of acid, HCO₃⁻ is secreted and pH in the lumen rises. When lumen pH rose above 7.4, another acid challenge was applied. After 15 to 30 min, when lumen pH responses had stabilized, one piece of mucosa was treated with R568 in the serosal bathing solution while the other piece of mucosa was treated with vehicle control, and changes in lumen pH responses during the 30-45 min period ensuing after the addition of the agonist were determined. Initial studies demonstrated that under these experimental conditions the tissue tolerated acid challenges for at least 60 minutes without significantly compromising tissue responses and integrity. Initial studies also established that acid-induced pH recovery reflects HCO₃⁻ secretion from tissue as additions of acid to no-tissue controls or tissue controls without the presence of serosal HCO₃⁻ did not generated significant pH recovery. A lowering of 0.3-0.4 pH units was used because this is the range of luminal pH that this part of the intestine normally varies. Initial rates of acid-induced pH recovery were used for comparison and were expressed as pH unit recovered/min/cm².

Secretagogue-Induced HCO₃⁻ Secretion

To examine CaSR effect on stimulated HCO₃⁻ secretion, a model of forskolin-induced HCO₃⁻ secretion was employed to mimic cholera toxin. The secretagogue forskolin was used to increase tissue cAMP content rapidly as cholera toxin's effect will not be seen for 1-2 hours. Tissues were divided and treated as in the basal HCO₃⁻ secretion experiments except that after 15 to 30 min, when I_{sc} and him had stabilized, forskolin was added to the serosal side of tissue for 15 to 30 min until I_{sc} and J_{HCO3} had plateaued before R568 was then added to the serosal solution. Changes in I_{sc} and J_{HCO3} during the 15-30 min period ensuing after the addition of the agonist were determined. Rodent distal colon displays Na⁺, K⁺ and Cl⁻ currents, in addition to HCO₃⁻ conductance. To reduce current interference from non HCO₃⁻ conductance, prior to the recording of HCO₃⁻ secretory responses, a cocktail containing 10 μM amiloride and 5 mM barium was added to the mucosal side to selectively inhibit Na⁺ and K⁺ conductance, and 100 μM bumetanide to the serosal side to inhibit Cl⁻ secretory current (26, 41). Preliminary studies indicate that these transport inhibitors did not significantly affect J_{HCO3} response although I_{sc} was inhibited by amiloride and stimulated by barium (see Table 3).

TABLE 3
Effect of transport inhibitors on basal
HCO₃⁻ secretion in rat distal colon

| | ΔJ_{HCO3} (μEq/hr/cm²) | ΔI_{sc} (μA/cm²) |
|---|---|---|
| Amiloride (10 μM, lumen) | 0.01 ± 0.01 (3)ns | −6.3 ± 1.0 (3)** |
| Barium (5 mM, lumen) | 0.02 ± 0.06 (3)ns | 4.7 ± 0.7 (3)** |
| Bumetanide (100 μM, | 0.03 ± 0.08 (3)ns | −2.3 ± 4.7 (3)ns |
| Serosa) | | |
| DIDS (100 μM, lumen) | −0.08 ± 0.04 (4)* | 4.6 ± 3.9 (4)ns |
| NPPB (100 μM, lumen) | −0.17 ± 0.06 (3)* | −10.2 ± 0.8 (3)** |

Cl⁻/HCO₃⁻ Exchange

Mucosa from each colon was divided longitudinally into 2 equal pieces and mounted into 2 Ussing chambers. One piece of mucosa was bathed luminally with zero HCO₃⁻ Ringer solution that contained Cl⁻ while the other piece of mucosa was bathed luminally with zero HCO₃⁻ Ringer solution that did not contain Cl⁻. Both pieces of mucosa were bathed serosally with Ringer solution that contained Cl⁻ and HCO₃⁻. After 15 to 30 min, when J_{HCO3} had stabilized, R568 or vehicle control was added to the serosal or mucosal side of tissue, and changes in J_{HCO3} during the 15-30 min period ensuing after the addition of the agonist or vehicle were determined and averaged. When inhibitor was used, it was added at 30 min before the agonist. Cl⁻/HCO₃⁻ exchange activity was calculated as ΔJ_{HCO3} (the difference between J_{HCO3} in the presence minus absence of luminal Cl⁻).

SCFA/HCO₃⁻ Exchange

Unless specifically described elsewhere, colon segments were prepared and treated as in the Cl⁻/HCO₃⁻ exchange studies except that they were bathed luminally with zero HCO₃⁻ Ringer solution that either contained or did not contain 25 mM isobutyrate. SCFA/HCO₃⁻ exchange activity was calculated as ΔJ_{HCO3} (the difference between J_{HCO3} in the presence minus absence of luminal isobutyrate).

Cyclic Nucleotide-Dependent HCO₃⁻ Secretion

Colon segments were prepared and treated as in the secretagogue-induced HCO₃⁻ secretion experiments except that they were bathed luminally with HCO₃⁻-free Ringer solution that contained no isobutyrate and no Cl⁻. Cyclic nucleotide-dependent HCO₃⁻ secretion was calculated as Ah_{ic03} (the difference between J_{HCO3} after minus J_{HCO3} before the addition of forskolin). When inhibitor was used, it was added at 30 min before R568 or forskolin.

### Chemicals and Solutions

Forskolin, 4,4′-diisothiocyano-2,2′-stilbenedisulfonic acid (DIDS), glibenclamide, 5-nitro-2-(3-phenylpropylamino)benzoic acid (NPPB), amiloride, barium, and bumetanide were obtained from Sigma and GlyH-101 from Santa Cruz Biotechnology while R-568 from Tocris Bioscience (Ellisville, Mich.). All stock solutions were prepared in DMSO. The detailed composition of Ringer solutions used in these studies is listed in Table 1. 5% CO₂ and 95% O₂ were used to oxygenate Ringer solutions that contained HCO₃⁻, while 100% O₂ was used to oxygenate those solutions that did not contain HCO₃⁻ Statistical Analysis

Values are expressed as means±SEM. ΔJ_{HCO3}, ΔI_{sc} and ΔpH recovery rates refer to stimulated peak responses minus basal control levels. Data were analyzed by one-way ANOVA followed by Holm-Sidak's post hoc test or, when appropriate, by the paired or unpaired two-tailed Student's t-test using Microsoft Excel 2010 for Windows or GraphPad Prism version 6 for Windows (GraphPad Software, San Diego, Calif.). p<0.05 was considered significant.

Following are examples which illustrate procedures for practicing the invention. These examples should not be construed as limiting. All percentages are by weight and all solvent mixture proportions are by volume unless otherwise noted.

### Example 1—Basal HCO₃⁻ Secretion

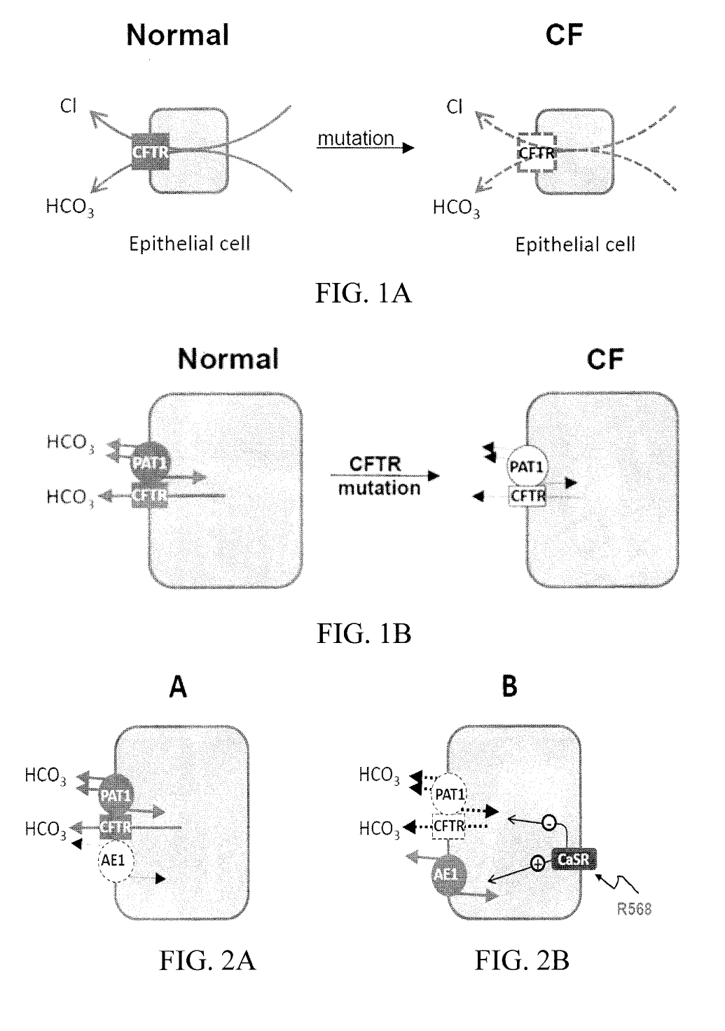

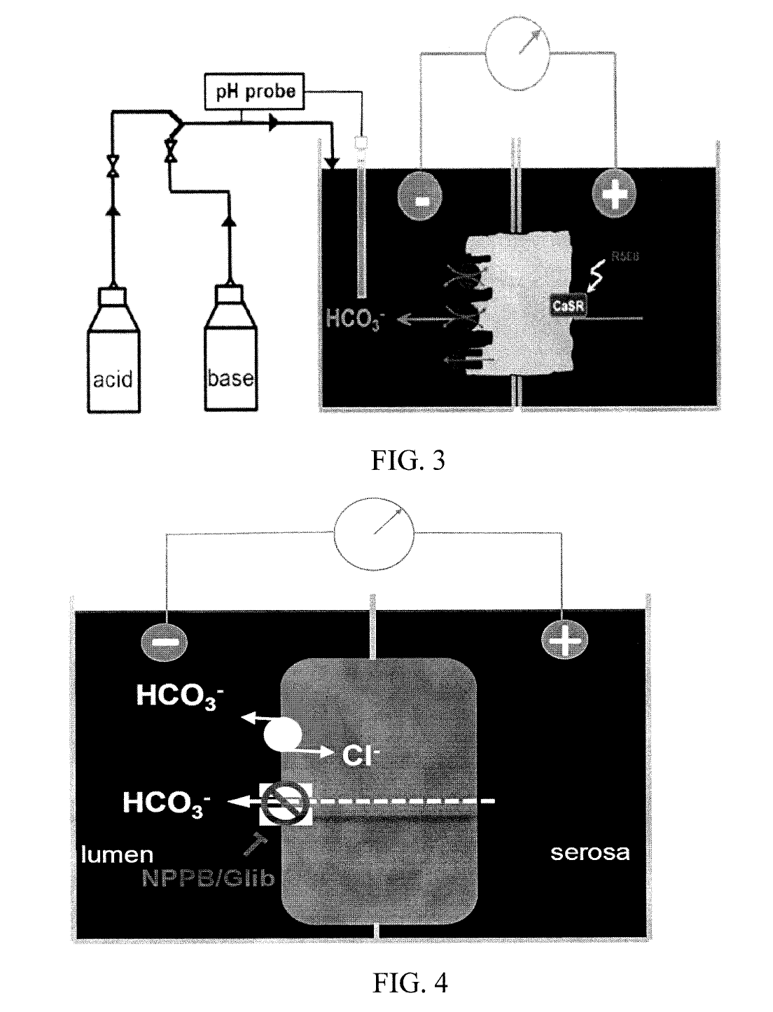

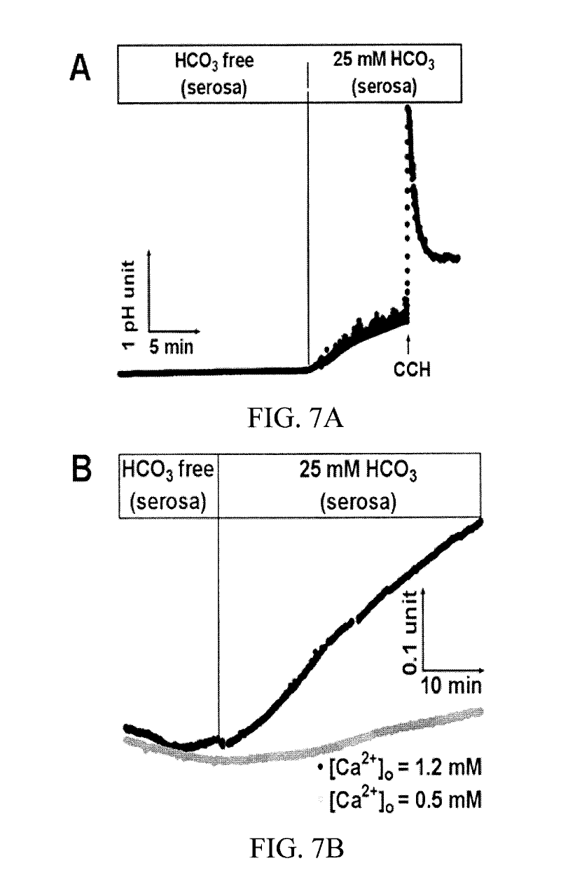

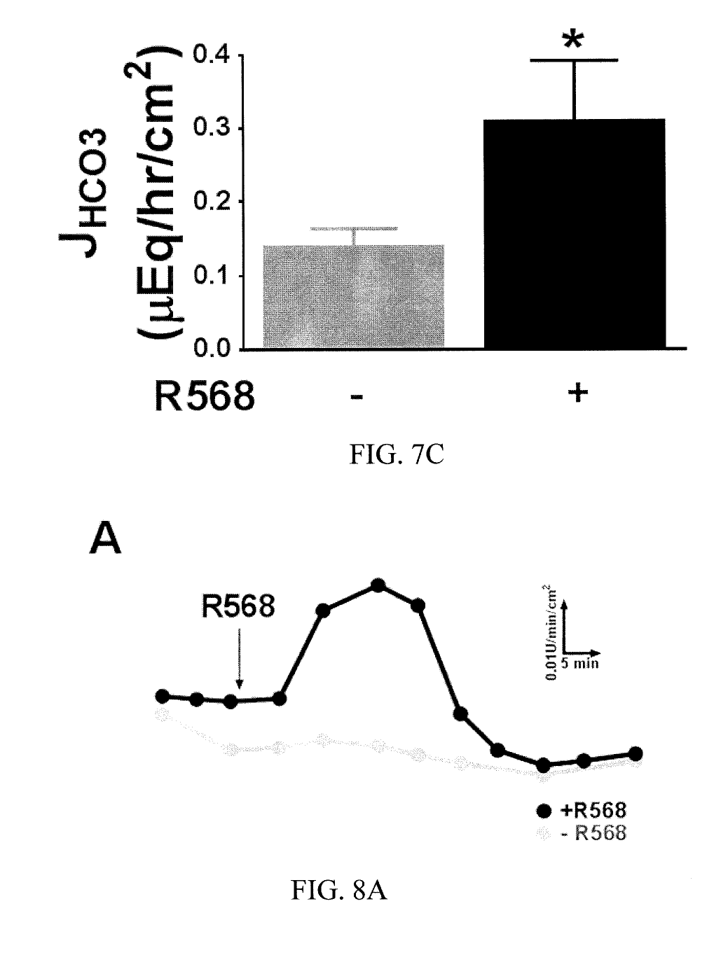

Lowering extracellular Ca²⁺ concentration reduced and activation of CaSR by R568 enhanced basal HCO₃⁻ secretion. The initial experiments performed examined the role of extracellular Ca²⁺ in modulation of HCO₃⁻ secretion under basal (non-stimulated) conditions. Mucosa was first bathed with HCO₃⁻ free, Cl⁻ Ringer solution before 25 mM HCO₃⁻ was added to the serosa. As shown in FIG. 7, there was no change in lumen pH; thus lumen alkalinization did not occur in the absence of a serosa-to-lumen directed HCO₃⁻ gradient. Consistent with HCO₃⁻ secretion, subsequent addition of HCO₃⁻ ion to serosal side induced significant lumen alkalinization [FIG. 7A; mean±SEM (n) absence vs. presence of HCO₃⁻ ion: −0.011±0.002 (3) vs. 0.035±0.002 (3) pH unit/min/cm², p<0.01]. Carbachol (CCH), a known secretagogue for HCO₃⁻ secretion was used as a positive control. CCH induced an initial transient increase followed by a sustained increase in lumen pH (FIG. 1A) characteristic of HCO₃⁻ secretion associated with cholinergic receptor activation.

Experiments were then performed to examine whether CaSR regulates basal HCO₃⁻ secretion. For this, the concentration of Ca²⁺ in the buffer was lowered from 1.2 mM to 0.5 mM and HCO₃⁻ secretory response compared. Extracellular Ca²⁺ is a physiological ligand of CaSR. Previous studies have shown that this maneuver reduces CaSR activity and that at 0.5 mM Ca²⁺ concentration CaSR is only minimally stimulated. Reduction of [Ca²⁺]ₒ from 1.2 mM to 0.5 mM significantly reduced lumen alkalinization rates [FIG. 1B; mean+SE (n) [Ca²⁺]ₒ=1.2 vs. 0.5 mM: 0.031±0.002 (3) vs. 0.013±0.002 (3) pH unit/min/cm², p<0.01]. As extracellular Ca²⁺ is also a known determinant of paracellular permeability of intestinal epithelium, additional experiments were performed to exclude the possibility that the reduced rate of lumen alkalinization noted at reduced [Ca²⁺] was not simply caused by HCO₃⁻ “back leak” secondary to altered paracellular integrity; thus, transepithelial electrical resistances (TEER) were measured. No significant differences in TEER were noted between normal and reduced Ca²⁺ treated tissues (see details in FIG. 7, legend). Thus, extracellular Ca²⁺ (and CaSR) may regulate transcellular, and not paracellular HCO₃⁻ movement.

To further assess the role of CaSR as a regulator of HCO₃⁻ secretion, another set of experiments measured the rate of serosal-to-mucosal HCO₃⁻ flux (J_{HCO3}) and HCO₃⁻ secretory I_{sc} and tested the effect of R568, a specific pharmacological CaSR agonist. A relatively low basal J_{HCO3} was observed in the absence of R568. Addition of R568 to serosa (FIG. 7C) significantly stimulated J_{HCO3}. Similar but slightly less pronounced effects were also noted when R568 was added to lumen solutions (not shown). Such stimulatory changes were not observed in I_{sc} [mean±SEM (n) absence vs. presence of R568: 1.36±0.40 (5) vs. 1.48±0.44. (5) μEq/hr/cm², p>0.05]. These data suggested that CaSR stimulates electroneutral HCO₃⁻ secretion.

### Example 2—Acid-Induced HCO₃⁻ Secretion

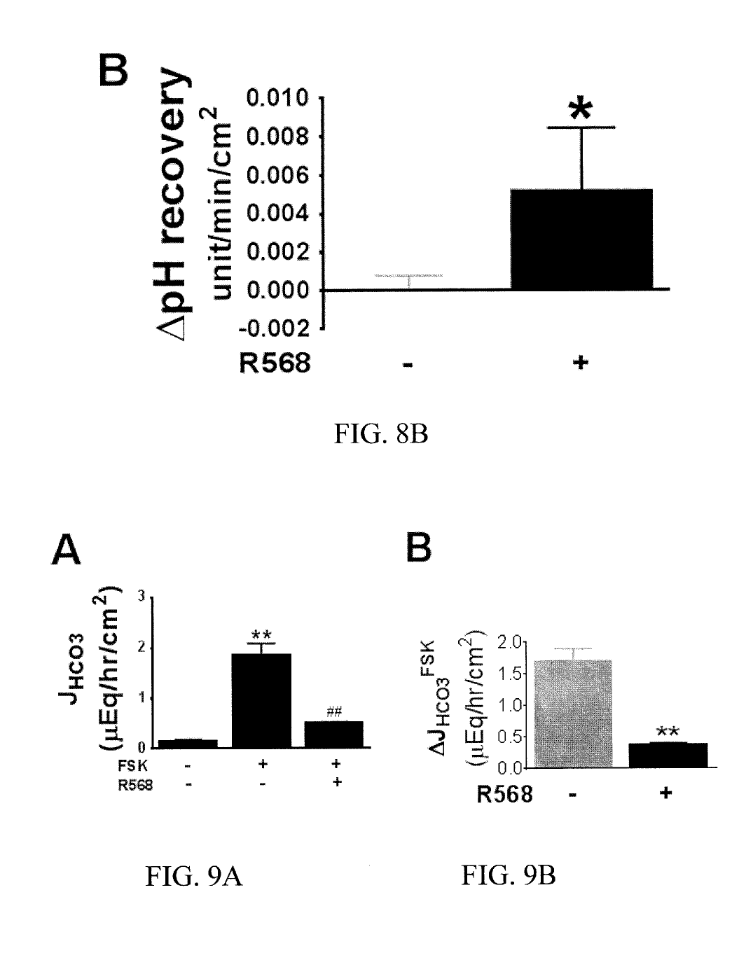

Activation of CaSR by R568 enhances acid-induced HCO₃⁻ secretion Acid-induced HCO₃⁻ secretion is a known mechanism that intestinal mucosa utilizes as a defense against acid-induced damage. Colonic mucosa is exposed to luminal acidic environment, generated as a result of bacteria fermentation of undigested carbohydrates. To assess if CaSR affects acid-induced HCO₃⁻ secretion, colonic tissues were challenged by additions of acid (HCl) to lower pH in the lumen, rates of pH recovery monitored and recorded, and effect of R568 examined. FIG. 8A shows changes in the rates of pH recovery in the presence vs. absence of R568. The \rate changes at peak responses above basal levels were calculated and are summarized in FIG. 8B. R568 induced a transient but significant stimulation of acid-induced HCO₃⁻ secretion.

### Example 3—Secretagogue-Induced HCO₃⁻ Secretion

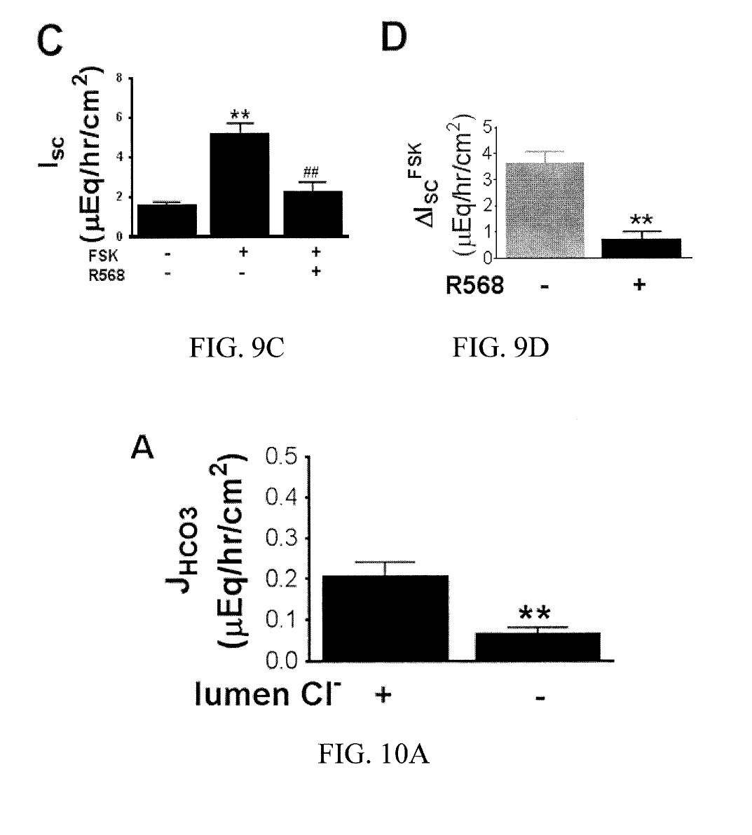

Activation of CaSR by R568 inhibits secretagogue-induced HCO₃⁻ secretion HCO₃⁻ secretion is markedly increased in cholera and other secretagogue-induced diarrheal diseases. To assess if CaSR stimulates HCO₃⁻ secretion under these diseased conditions, the R568 effect was examined in a model of secretagogue-induced secretory diarrhea. Forskolin was used to stimulate HCO₃⁻ secretion, and HCO₃⁻ secretory response was monitored by measuring HCO₃⁻ secretory rate (J_{HCO3}) (FIG. 9A) and by recording I_{SS} (FIG. 9B). Forskolin stimulated both J_{HCO3} and I_{SC}; subsequent addition of R568 did not increase but rather decreased forskolin-induced HCO₃⁻ secretion.

### Example 4—Cl⁻ Dependent HCO₃⁻ Secretion

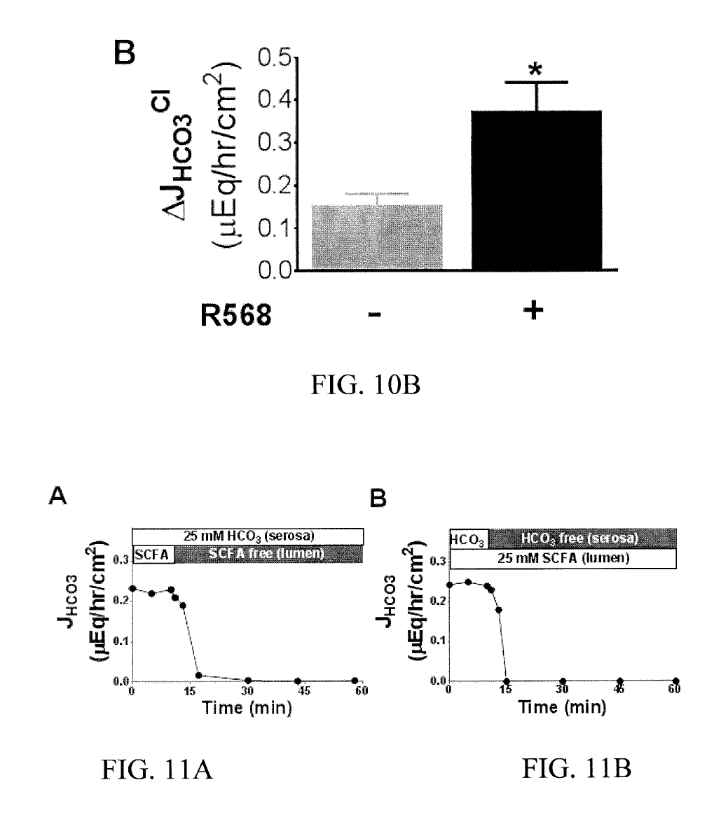

Activation of CaSR stimulates Cl⁻/HCO₃⁻ exchange activity Since the basal and acid-induced HCO₃⁻ secretion were assayed in Cl⁻-containing Ringer, it is likely that the CaSR effects reflect stimulation of a Cl⁻-dependent HCO₃⁻ secretory mechanism such as Cl⁻/HCO₃⁻ exchange. To address this possibility, Cl⁻/HCO₃⁻ exchange activity was measured and is shown in FIG. 10. FIG. 10A demonstrates HCO₃⁻ secretory responses to the presence vs. absence of lumen Cl⁻. A luminal Cl⁻-dependent HCO₃⁻ secretory mechanism (Cl⁻/HCO₃⁻ exchange) was observed. The latter was partially abolished by pretreatment with 100 μM DIDS added to lumen side (data not shown). Activation of CaSR by R568, added to serosal side, resulted in stimulation of the Cl⁻/HCO₃⁻ exchange activity (FIG. 10B). This CL-dependent HCO₃⁻ secretion was significantly higher in the presence than in the absence of R568 (FIG. 10B). A similar but less pronounced stimulatory effect of R568 was noted when this agonist was added to the mucosal solution (data not shown).

### Example 5—SCFA-Dependent HCO₃⁻ Secretion

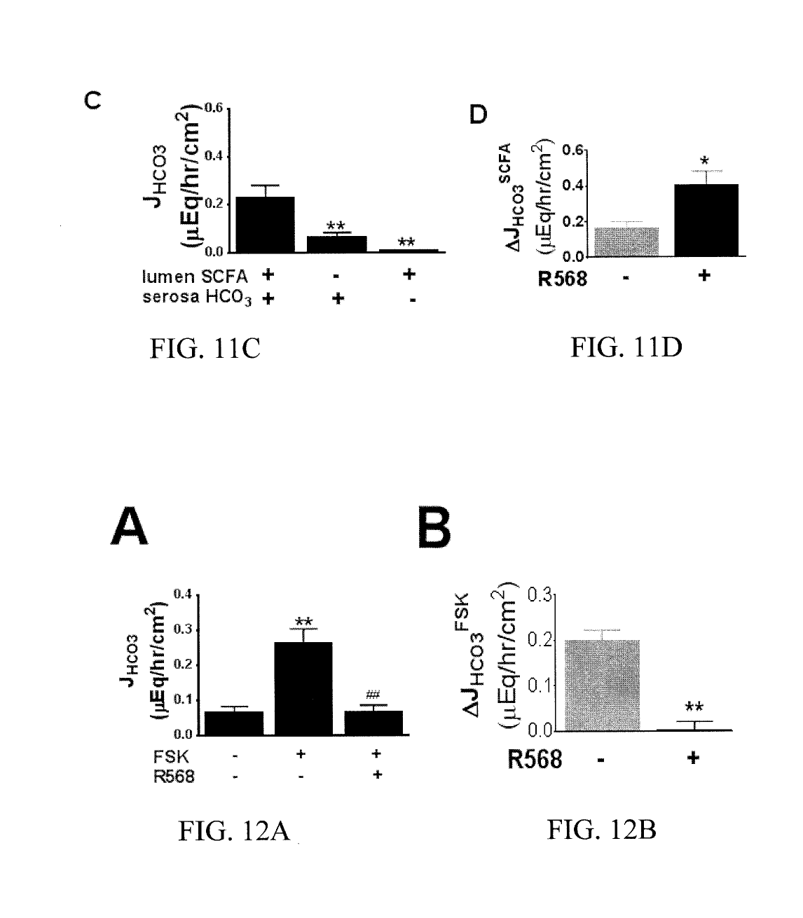

Activation of CaSR stimulates SCFA/HCO₃⁻ exchange activity—Short-chain fatty acids (SCFA) are present in the colon and induce HCO₃⁻ secretion via the SCFA/HCO₃⁻ exchange. To assess if CaSR stimulates HCO₃⁻ secretion under these conditions, the next series of experiments examined isobutyrate-dependent HCO₃⁻ secretion and its response to R568. FIGS. 11A & 11B show representative tracings and FIG. 11C presents a summary of HCO₃⁻ secretory responses to the presence vs. absence of lumen SCFA (25 mM isobutyrate) with vs. without a serosa-to-mucosa directed HCO₃⁻ gradient. SCFA-dependent HCO₃⁻ secretion was present and required serosal HCO₃⁻, consistent with HCO₃⁻ secretion mediated by SCFA/HCO₃⁻ exchange. Similar to the R568 effect on the Cl⁻/HCO₃⁻ exchange, isobyturate-dependent HCO₃⁻ secretion was significantly stimulated by activation of CaSR by R568 (FIG. 11D).

### Example 6—Camp-Dependent HCO₃⁻ Secretion

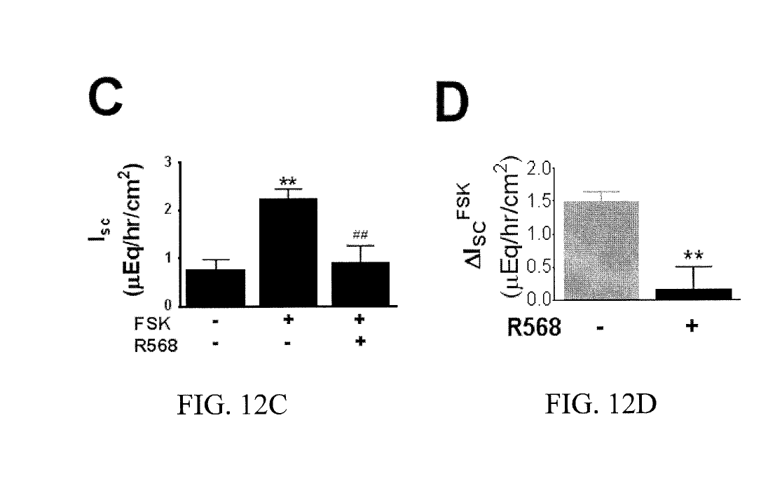

Activation of CaSR by R568 inhibits cAMP-dependent HCO₃⁻ secretion—In contrast to R568 stimulation of basal HCO₃⁻ secretion, HCO₃⁻ secretion was inhibited by R568 under forskolin-stimulated condition (FIG. 9). It is uncertain how R568 produces this inhibition. Since R568 inhibited neither Cl⁻-dependent nor SCFA-dependent HCO₃⁻ secretion (FIGS. 10-11), it is unlikely that the effect of the CaSR agonist is via inhibition of either of these anion exchanges. Rather, a Cl⁻/SCFA-independent HCO₃⁻ transport mechanism(s) might be responsible. One such mechanism is a cyclic nucleotide-dependent electrogenic channel (e.g., CFTR)-mediated HCO₃⁻ secretion. Thus, to address this latter possibility, stimulation of HCO₃⁻ secretion by forskolin and its inhibition following addition of R568 were re-assessed in lumen Cl⁻/SCFA-free solutions. As shown in FIG. 12A, a low rate of HCO₃⁻ transport was noted under basal condition prior to the addition of forskolin. Addition of forskolin significantly stimulated HCO₃⁻ secretion. The subsequent addition of R568 almost completely reversed FSK-induced HCO₃⁻ secretion.

Similar changes were observed in experiments that determined changes in I_{sc} (FIG. 12B). Forskolin stimulated I_{sc}; subsequent addition of R568 inhibited HCO₃⁻ secretion. Removal of the serosa-to-mucosa HCO₃⁻ gradient significantly diminished both basal and forskolin-stimulated I_{sc} (data not shown); in the absence of serosal HCO₃⁻, forskolin and R568 failed to stimulate or to inhibit I_{sc}, respectively (data not shown). Pretreatment with luminal glibenclamide (100 μM) or GlyH-101 (10 μM), CFTR channel blockers, and NPPB (100 μM), an anion channel inhibitor, added either before the addition of forskolin or R568, abolished the forskolin stimulatory and R568 inhibitory effects on I_{sc} (data not shown). As a consequence, these results suggest that R568 inhibits cAMP-dependent, glibenclamide/GlyH-101/NPPB-sensitive electrogenic HCO₃⁻ secretion.

### Example 7—Effect of CaSR Knock-Out

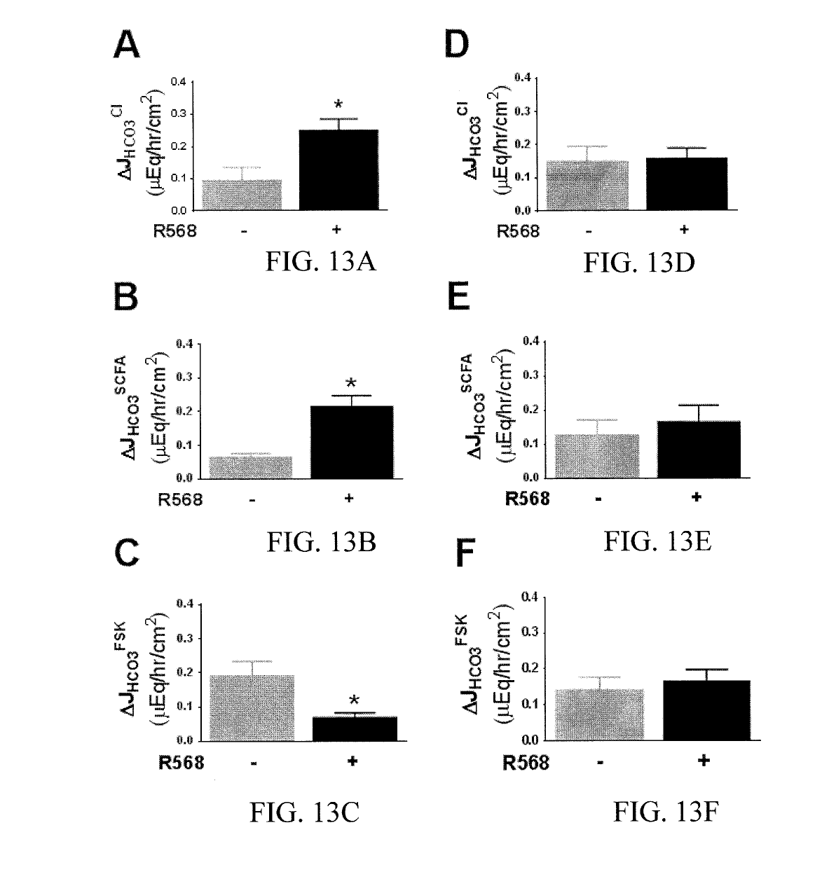

R568 fails to stimulate Cl⁻- and SCFA-dependent and inhibit cAMP-dependent HCO₃⁻ secretion in colon of CaSR null mouse—R568 is a specific pharmacological agonist that has been widely used to stimulate CaSR. To verify that the effects of R568 occurred via the CaSR, additional studies on the effect of R568 were performed in intestinal epithelium-specific CaSR knockout mice (FIG. 13). Intestinal epithelium-specific CaSR knockout mice were used together with their wild type littermates. Activation of CaSR by R568 stimulated Cl⁻- and SCFA-dependent HCO₃⁻ secretion and inhibited cAMP-dependent HCO₃⁻ secretion in colon mucosa of wild type mice (FIG. 13A-C); such effects were abolished in CaSR null mice (FIG. 13D-F). These results indicate that the R568 effects occur via activating the CaSR in the intestinal epithelium.

### Example 8—Regulation of HCO₃⁻ Secretion in the Colon

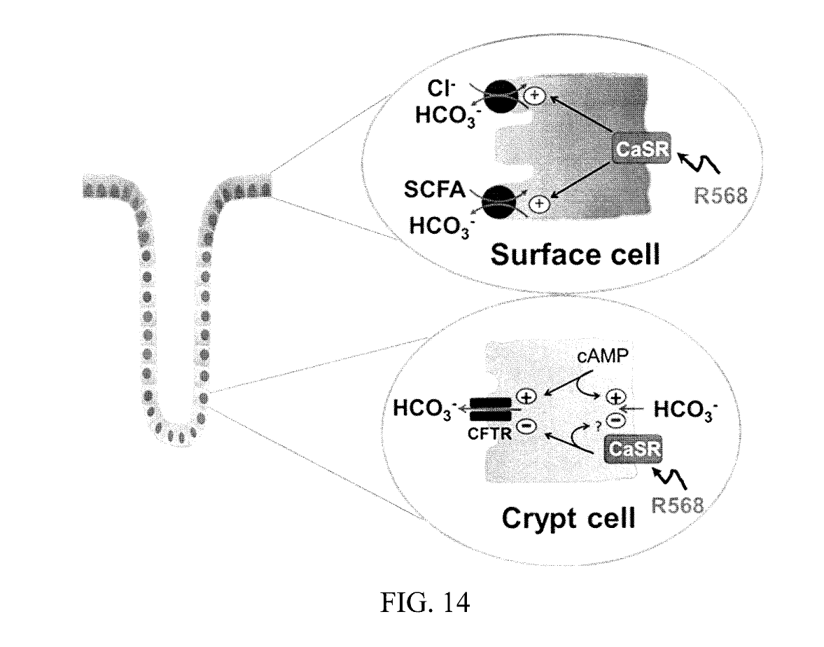

The current invention provides a new model for regulation of HCO₃⁻ secretion in the mammalian colon where activation of CaSR by R568 stimulated basal and acid-induced HCO₃⁻ secretion but, in contrast, R568 inhibited cyclic nucleotide-mediated HCO₃⁻ secretion. The invention also indicates that the enhancement of HCO₃⁻ secretion is mediated via stimulation of electroneutral Cl⁻/HCO₃⁻ and SCFA/HCO₃⁻ exchanges that are localized on the apical membrane of colonic surface epithelial cells; in contrast, the CaSR inhibitory action is a consequence of CaSR inhibition of a cAMP-dependent, lumen glibenclamide/GlyH-101/NPPB-sensitive electrogenic HCO₃⁻ secretory process primarily located in the crypt cells. A model for this differential regulation of colonic HCO₃ secretion by CaSR is depicted in FIG. 14.

According to the present model of colonic ion function, absorptive processes are primarily localized to surface cells whereas secretory processes are primarily present in crypt cells. Because Cl⁻-dependent HCO₃⁻ exchange and SCFA-dependent HCO₃⁻ exchange are present only in surface cells and are absent in crypts, Cl⁻-dependent HCO₃⁻ secretion and SCFA-dependent HCO₃⁻ secretion are most likely both surface cell functions. In contrast, CFTR-mediated HCO₃⁻ secretion is generally considered to represent a crypt cell function. Thus, in addition to mediating colonic HCO₃⁻ secretion, the primary function of these two anion exchanges in these absorptive surface cells is to absorb solutes/electrolytes. SCFA absorption is mediated by SCFA/HCO₃⁻ exchange, and NaCl absorption is the result of Cl⁻/HCO₃⁻ exchange coupled to Na⁺/H⁺ exchange, which is also localized in surface cells. The ability of CaSR agonists to stimulate both anion exchanges (FIGS. 10-13) as well as Na⁺/H⁺ exchange suggests that, in addition to stimulation of HCO₃⁻ secretion, CaSR may function as a mechanism to enhance electrolyte and fluid absorption. Activation of CaSR also stimulates colonic acid-induced HCO₃⁻ secretion (FIG. 8). This latter function may also neutralize H⁺ from bacterial fermentation and/or Na⁺/H⁺ exchange, further increasing solute absorption.

Consistent with a recent in vivo study in rat perfused duodenum, the invention demonstrated that CaSR activation stimulated basal HCO₃⁻ secretion in ex vivo colonic mucosa. These findings may have important physiological significance as HCO₃⁻ secretion is an integral part of mucosal defense mechanisms. HCO₃⁻ secretion is required for mucin secretion by goblet cells to establish a layer of mucus overlying the epithelium, an initial defense barrier that limits pathogen invasion. Defects in HCO₃⁻ secretion have been shown to impair the formation of the mucus layer and compromise the integrity of the intestinal barrier, leading to bacteria translocation and development of intestinal inflammation. Thus, the ability for CaSR to stimulate HCO₃⁻ secretion under basal conditions suggests that, through modulating mucus secretion and barrier function, this well-conserved nutrient-sensing receptor may play a role in intestinal immune function. Indeed, mice deficient in CaSR with deficiently regulated HCO₃⁻ secretion in the colon have altered barrier integrity, enhanced bacteria translocation and increased inflammation whereas enteral nutrients, including the CaSR-activating nutrients/minerals, calcium, spermine and tryptophan, have been shown to improve intestinal permeability and immunity and inflammation.

Importantly, both HCO₃⁻ and Cl⁻ secretion are markedly induced in cyclic nucleotide-mediated secretory diarrheas (e.g., cholera). Although these secretory responses may be helpful in enhancement of the defensing mucus layer so as to limit pathogen invasion and also to flush out toxins, over production and secretion of these anions by the intestine under these pathological conditions is harmful and may result in dehydration, alkali deficit and metabolic acidosis. Systemic volume depletion and metabolic acidosis are the two major causes of death associated with acute diarrheal illnesses, especially in infants and young children. The ability of CaSR agonists both to inhibit cyclic nucleotide-stimulated Cl⁻ and HCO₃⁻ secretion (FIGS. 12 & 13) and to promote Cl⁻ and SCFA absorption (FIGS. 10-13) as well as Na⁺ absorption suggests that this class of drugs may provide a unique therapeutic approach to prevent and treat these potentially lethal diarrheal illnesses. Since CaSR agonists are naturally occurring nutrients, CaSR-based anti-diarrheal therapies would be of particular utility among actively growing infants and children.

The net increases in I_{SS} induced by forskolin (ΔI_{sc}^{FSK}) were greater than those in net J_{FIC03} (ΔJ_{HCO3}^{FSK}) [compare the grey-colored columns in FIGS. 9D and 12D (mean values: 3.6 and 1.5 μEq/hr/cm²) vs. FIGS. 9B and 12B (mean values: 1.7 and 0.2 μEq/hr/cm²)]. These differences cannot be explained by a non-steady-state flux period as all measurements were made after 15-30 minutes when both I_{sc} and J_{HCO3} had stabilized and were in steady state. The most likely explanation is that a component of ΔI_{sc}^{FSK} represents forskolin-induced Cl⁻ secretion even though serosal bumetanide was present in both experiments. Serosal bumetanide was employed to prevent (or at least to reduce) such a contribution from forskolin-induced Cl⁻ secretion. It is known that in rat distal colon bumetanide does not completely suppress Cl⁻ secretion induced by forskolin. Only ˜70% of such Cl⁻ secretion was inhibited by bumetanide; the remainder of the Cl⁻ secretion was mediated by a Cl⁻/HCO₃⁻ exchange located in the basolateral membrane of colonocytes. Consistent with this, we found that the non-HCO₃⁻ portion of ΔI_{sc}^{FSK} was greater when a serosa-to-mucosa transepithelial Cl⁻ gradient was present than when a Cl⁻ gradient was absent (compare 88% in FIG. 12 vs. 53% in FIG. 9).

Although most experiments were performed with a reduced concentration of Ca²⁺ₒ in order to minimize background activation of the receptor before R568 addition, under normal Ca²⁺ₒ condition the effects of R568 were qualitatively similar, albeit with a slightly less pronounced effect. As such, the invention provides that the calcimimetic R568 has physiological relevance and clinical utility. Indeed, this same class of drug has been employed successfully to inhibit parathyroid hormone secretion in hyperparathyroid patients, where Ca²⁺ₒ in the serum can be either <1.0 mM (secondary hyperparathyroidism) or >1.5 mM (primary hyperparathyroidism).

In summary, the present invention confirms the presence of at least three distinct mechanisms for HCO₃⁻ secretion in rodent distal colon, i.e., lumen Cl⁻-dependent HCO₃⁻ secretion, SCFA-dependent HCO₃⁻ secretion, and cAMP-activated HCO₃⁻ secretion. Further, CaSR agonists differently regulate HCO₃⁻ secretion, depending on the physiological state of the intestine and the specific transporter in question. During physiological conditions when electroneutral Cl⁻/HCO₃⁻ and SCFA/HCO₃⁻ exchanges dominate, CaSR enhances HCO₃⁻ secretion; however, in experimental conditions that result in stimulation of fluid and HCO₃⁻ secretion that also occurs in cholera in which electrogenic CFTR-mediated HCO₃⁻ conductance is dominant, CaSR also inhibits HCO₃⁻ secretion. Both of these regulatory processes induced by CaSR are potentially beneficial. While the stimulatory effect may help expand the mucus layer, the inhibition of channel-mediated HCO₃⁻ secretion may be of particular clinical significance as it may reduce and minimize HCO₃⁻ losses in diarrhea.

### Example 9—Correction of Cation Transport in CF by R568

In addition to defects in anion transport, patients with CF also exhibit a defect in cation transport. Adequate luminal hydration required for the maintenance of health in all transporting epithelia is achieved by a fine balance between the two interconnected transport processes; the apical CFTR, which secretes anions (Cl⁻ and HCO₃⁻) and the Epithelian Sodium Channel (ENaC), which absorbs cation (Na⁺).

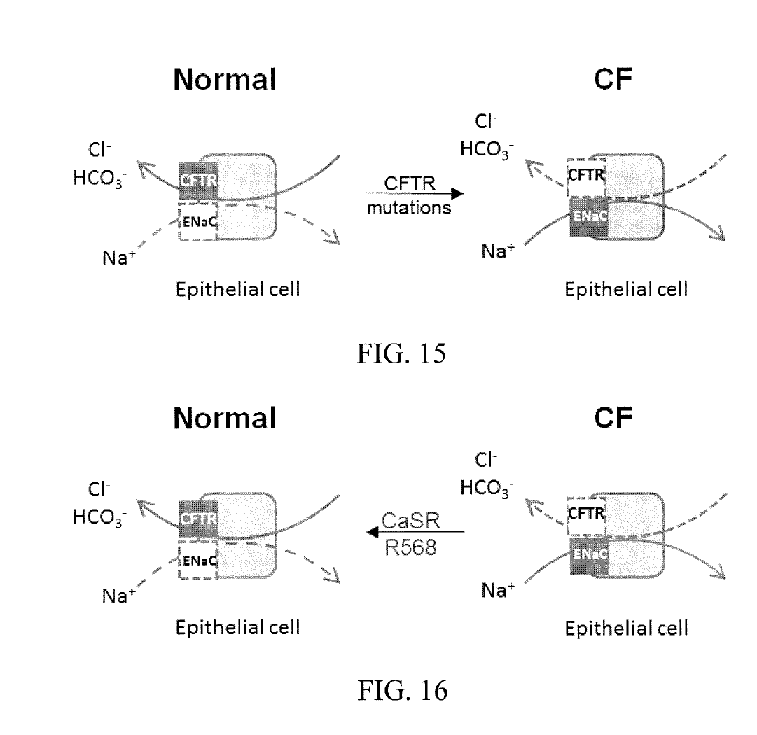

Normally, ENaC is inhibited by CFTR. However, in CF, CFTR loses this regulatory function. As a consequence, ENaC becomes overly activated and transepithelial Na⁺ and water is inappropriately absorbed and the lumen becomes inadequately hydrated and mucus plugging is formed (FIG. 15).

Therefore, inhibiting ENaC-mediated Na⁻ absorption is critical in prevention and treatment of CF. This hypothesis, depicted in FIG. 16, was examined by measuring changes in ENaC current with Ussing chamber-short circuit current (I_{sc}) recording (which measures the electrogenic Na⁺ absorption mediated by ENaC) in conjunction with the use of bumetanide (which specifically inhibits transepithelial Cl⁻ secretion mediated by CFTR and NKCCl).

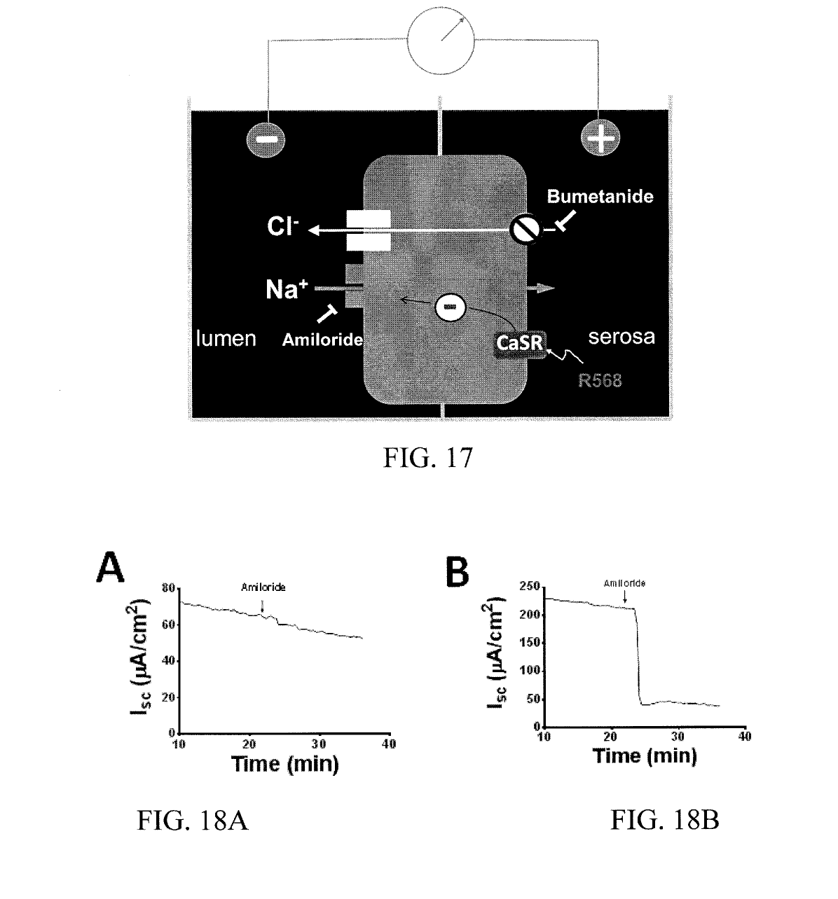

The experimental setup is illustrated in FIG. 17. In the distal colon of 2-3 week old weanling rats, the short-circuit current (Isc) is primarily made up by the following two electrogenic ion transport processes: 1) secretory Cl current, and 2) absorptive Na current. Secretory Cl current is mediated by apical CFTR. This Cl secretory Isc is sensitive to bumetanide, applied to the basolateral side of the epithelium. There, bumetanide inhibits Cl entry from blood into the cell; thus, in the presence of bumetanide, the secretory Cl current is eliminated. Absorptive Na current is mediated by apically located epithelial Na channel (ENaC). This latter current is particularly high in this weanling age and is sensitive to amiloride, when it is applied luminally.

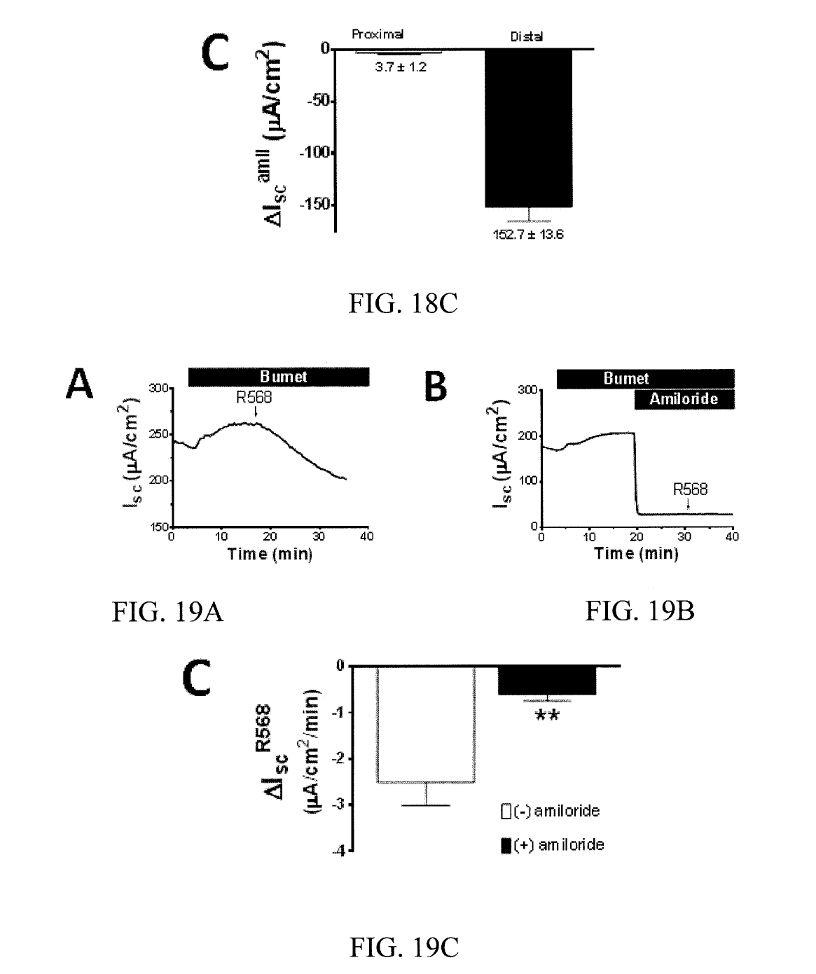

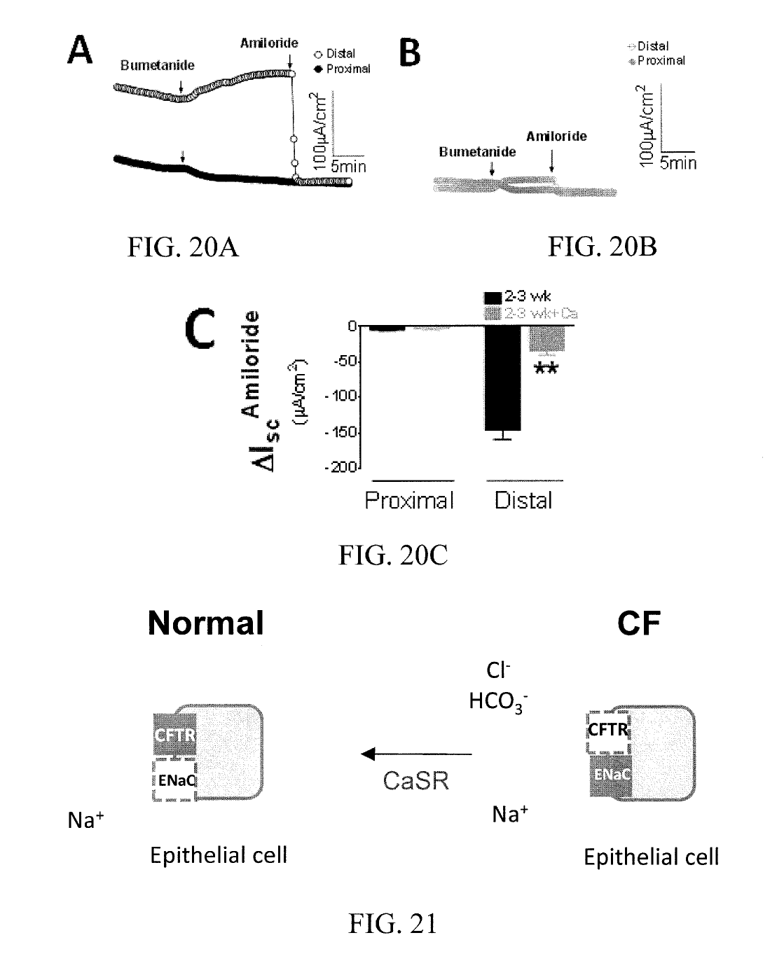

The basal I_{sc} in distal colon of weanling infant was mostly due to amiloride-sensitive Na⁺ absorption mediated by ENaC (FIG. 18). Activation of CaSR by R568 inhibited the bumetanide-insensitive amiloride-inhibitable ENaC-mediated basal I_{sc} in the distal colon of weanling infant (FIG. 19). Activation of CaSR by calcium (2.5% w/w, added to diet) suppressed the amiloride-sensitive ENaC-mediated basal I_{sc} in distal colon of weanling infant (FIG. 20). Therefore, activated CaSR inhibits ENaC activity. Hence, CaSR agonists can be used for correcting CF-associated defects in cation transport (FIG. 21).

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

### REFERENCES

1. Akiba Y, Watanabe C, Mizumori M, and Kaunitz J D. Luminal L-glutamate enhances duodenal mucosal defense mechanisms via multiple glutamate receptors in rats. American journal of physiology Gastrointestinal and liver physiology 297: G781-791, 2009.
2. Allen A, and Flemstrom G. Gastroduodenal mucus bicarbonate barrier: protection against acid and pepsin. American journal of physiology Cell physiology 288: C1-19, 2005.
3. Binder H J, Rajendran V, Sadasivan V, and Geibel J P. Bicarbonate secretion: a neglected aspect of colonic ion transport. Journal of clinical gastroenterology 39: S53-58, 2005.
4. Black R, Cousens S, Johnson H, Lawn J, Rudan I, Bassani D, Jha P, Campbell H, Walker C, Cibulskis R, Eisele T, Liu L, and Mathers C. Global, regional, and national causes of child mortality in 2008: a systematic analysis. Lancet 375: 1969-1987, 2008.
5. Bovee-Oudenhoven I M J, Lettink-Wissink M L G, Van Doesburg W, Witteman B J M, and Van Der Meer R. Diarrhea caused by enterotoxigenic Escherichia coli infection of humans is inhibited by dietary calcium. Gastroenterology 125: 469-476, 2003.
6. Bovee Oudenhoven I M, Wissink M L, Wouters J T, and Van der Meer R. Dietary calcium phosphate stimulates intestinal lactobacilli and decreases the severity of a salmonella infection in rats. The Journal of nutrition 129: 607-612, 1999.
7. Brown E M, Gamba G, Riccardi D, Lombardi M, Butters R, Kifor O, Sun A, Hediger M A, Lytton J, and Hebert S C. Cloning and characterization of an extracellular Ca(2+)-sensing receptor from bovine parathyroid. Nature 366: 575-580, 1993.
8. Brown E M, and MacLeod RJ. Extracellular calcium sensing and extracellular calcium signaling. Physiol Rev 81: 239-297, 2001.
9. Chattopadhyay N, Cheng I, Rogers K, Riccardi D, Hall A, Diaz R, Hebert S C, Soybel D I, and Brown E M. Identification and localization of extracellular Ca(2+)-sensing receptor in rat intestine. The American journal of physiology 274: G122-130, 1998.
10. Cheng C Y, Petrova E, Stahl M, and Cheng S X. Calcium-sensing receptor inhibits cholera toxin-induced anion secretion by intestine via enteric nervous system (Abstract). JPGN 55: E20, 2012.
11. Cheng S X. Calcium-sensing receptor in the gut: evidence for its role in mediating known nutritional therapy for inflammatory bowel disease (abstract). JPGN 55: E70, 2012.
12. Cheng S X. Calcium-sensing receptor inhibits secretagogue-induced electrolyte secretion by intestine via the enteric nervous system. The American journal of physiology 303: G60-G70, 2012.
13. Cheng S X, Bai H X, Gonzalez-Peralta R, Mistry P K, and Gorelick F S. Calcium ameliorates diarrhea in immunocompromised children. Journal of pediatric gastroenterology and nutrition 56: 641-644, 2013.
14. Cheng S X, Geibel J, and Hebert S. Extracellular polyamines regulate fluid secretion in rat colonic crypts via the extracellular calcium-sensing receptor. Gastroenterology 126: 148-158, 2004.
15. Cheng S X, Lightfoot Y L, Yang T, Zadeh M, Tang L, Sahay B, Wang G P, Owen J L, and Mohamadzadeh M. Epithelial CaSR Deficiency Alters Intestinal Integrity and Promotes Proinflammatory Immune Responses. FEBS Lett May 17. pii: S0014-5793(0014)00368-00368. doi: 00310.01016/j.febslet.02014.00305.00007, 2014.
16. Cheng S X, Okuda M, Hall A, Geibel J P, and Hebert S C. Expression of calcium-sensing receptor in rat colonic epithelium: evidence for modulation of fluid secretion. The American journal of physiology 283: G240-250, 2002.
17. Currie M G, Fok K F, Kato J, Moore R J, Hamra F K, Duffin K L, and Smith C E. Guanylin: an endogenous activator of intestinal guanylate cyclase. Proc Natl Acad Sci USA 89: 947-951, 1992.
18. Dohgen M, Hayashi H, Yajima T, and Suzuki Y. Stimulation of bicarbonate secretion by luminal short-chain fatty acid in the rat and human colon in vitro. Jpn J Physiol 44: 519-531, 1994.
19. Field M, Graf L H, Laird W J, and Smith P L. Heat-stable enterotoxin of Escherichia coli: in vitro effects on guanylate cyclase activity, cyclic GMP concentration, and ion transport in small intestine. Proc Natl Acad Sci USA 75: 2800-2804, 1978.
20. Flemstrom G, and Isenberg J I. Gastroduodenal mucosal alkaline secretion and mucosal protection. News in physiological sciences an international journal of physiology produced jointly by the International Union of Physiological Sciences and the American Physiological Society 16: 23-28, 2001.
21. Flores J, and Sharp G. The activation of adenylate cyclase by cholera toxin: possible interaction with the nucleotide regulatory site. Ciba Found Symp 42: 89-108, 1976.
22. Fordtran J S. Speculations on the pathogenesis of diarrhea. Federation proceedings 26: 1405-1414, 1967.
23. Gama L, Baxendale-Cox L M, and Breitwieser G E. Ca2+-sensing receptors in intestinal epithelium. The American journal of physiology 273: C1168-1175, 1997.
24. Garcia M A, Yang N, and Quinton P M. Normal mouse intestinal mucus release requires cystic fibrosis transmembrane regulator-dependent bicarbonate secretion. The Journal of clinical investigation 119: 2613-2622, 2009.
25. Geibel J, Sritharan K, Geibel R, Geibel P, Persing J S, Seeger A, Roepke T, Deichstetter M, Prinz C, Cheng S, Martin D, and Hebert S. Calcium-sensing receptor abrogates secretagogue-induced increases in intestinal net fluid secretion by enhancing cyclic nucleotide destruction. Proc Natl Acad Sci USA 103: 9390-9397, 2006.
26. Haas M, and McManus T J. Bumetanide inhibits (Na/K/2C1) co-transport at a chloride site. The American journal of physiology 245: C235-240, 1983.
27. Holmberg C, Perheentupa J, and Launiala K. Colonic electrolyte transport in health and in congenital chloride diarrhea. The Journal of clinical investigation 56: 302-310, 1975.
28. Isenberg J I, Selling J A, Hogan D L, and Koss M A. Impaired proximal duodenal mucosal bicarbonate secretion in patients with duodenal ulcer. The New England journal of medicine 316: 374-379, 1987.
29. Liou A, Sei Y, Zhao X, Feng J, Lu X, Thomas C, Pechhold S, Raybould H, and Wank S. The extracellular calcium-sensing receptor is required for cholecystokinin secretion in response to L-phenylalanine in acutely isolated intestinal I cells. American journal of physiology: Gastrointestinal and liver physiology 300: G538-546, 2011.
30. Macleod R J. Extracellular calcium-sensing receptor/PTH knockout mice colons have increased Wnt/β-catenin signaling, reduced non-canonical Wnt signaling, and increased susceptibility to azoxymethane-induced aberrant crypt foci. Lab Invest 93: 520-527, 2013.
31. Moore S, Lima A A M, and Guerrant R. Infection: Preventing 5 million child deaths from diarrhea in the next 5 years. Nature Rev Gastroenterol Hepatol 8: 363-364, 2011.
32. Nearing J, Betka M, Quinn S, Hentschel H, Elger M, Baum M, Bai M, Chattopadyhay N, Brown E M, Hebert S C, and Harris H W. Polyvalent cation receptor proteins (CaRs) are salinity sensors in fish. Proceedings of the National Academy of Sciences of the United States of America 99: 9231-9236, 2002.
33. Nugent S G, Kumar D, Rampton D S, and Evans D F. Intestinal luminal pH in inflammatory bowel disease: possible determinants and implications for therapy with aminosalicylates and other drugs. Gut 48: 571-577, 2001.
34. Powell D W, Solberg L I, Plotkin G R, Catlin D H, Maenza R M, and Formal S B. Experimental diarrhea. 3. Bicarbonate transport in rat salmonella enterocolitis. Gastroenterology 60: 1076-1086, 1971.
35. Quinton P M. Role of epithelial HCO3(−) transport in mucin secretion: lessons from cystic fibrosis. American journal of physiology Cell physiology 299: C1222-1233, 2010.
36. Rey O C W, Bikle D, Rozengurt N, Young S H, Rozengurt E. Negative cross-talk between calcium-sensing receptor and β-catenin signaling systems in colonic epithelium. The Journal of biological chemistry 287: 1158-1167, 2012.
37. Riccardi D, Park J, Lee W S, Gamba G, Brown E M, and Hebert S C. Cloning and functional expression of a rat kidney extracellular calcium/polyvalent cation-sensing receptor. Proceedings of the National Academy of Sciences of the United States of America 92: 131-135, 1995.
38. Sands J M, Naruse M, Baum M, Jo I, Hebert S C, Brown E M, and Harris H W. Apical extracellular calcium/polyvalent cation-sensing receptor regulates vasopressin-elicited water permeability in rat kidney inner medullary collecting duct. J Clin Invest 99: 1399-1405, 1997.
39. Schepens M A, Rijnierse A, Schonewille A J, Vink C, Brummer R J, Willemsen L E, van der Meer R, and Bovee-Oudenhoven I M. Dietary calcium decreases but short-chain fructo-oligosaccharides increase colonic permeability in rats. The British journal of nutrition 104: 1780-1786, 2010.
40. Schepens M A, Vink C, Schonewille A J, Dijkstra G, van der Meer R, and Bovee-Oudenhoven I M. Supplemental calcium attenuates the colitis-related increase in diarrhea, intestinal permeability, and extracellular matrix breakdown in HLA-B27 transgenic rats. J Nutr 139: 1525-1533, 2009.
41. Schultheiss G, Horger S, and Diener M. The bumetanide-resistant part of forskolin-induced anion secretion in rat colon. Acta physiologica Scandinavica 164: 219-228, 1998.
42. Seamon K B, and Daly J W. Forskolin: a unique diterpene activator of cyclic AMP-generating systems. J Cyclic Nucleotide Res 7: 201-224, 1981.
43. Seidler U, Blumenstein I, Kretz A, Viellard-Baron D, Rossmann H, Colledge W H, Evans M, Ratcliff R, and Gregor M. A functional CFTR protein is required for mouse intestinal cAMP-, cGMP- and Ca(2+)-dependent HCO3-secretion. The Journal of physiology 505: 411-423, 1997.
44. Sheinin Y, Kallay E, Wrba F, Kriwanek S, Peterlik M, and Cross H S. Immunocytochemical localization of the extracellular calcium-sensing receptor in normal and malignant human large intestinal mucosa. J Histochem Cytochem 48: 595-602, 2000.
45. Talbot C, and Lytle C. Segregation of Na/H exchanger-3 and Cl/HCO3 exchanger SLC26A3(DRA) in rodent cecum and colon. American journal of physiology Gastrointestinal and liver physiology 299: G358-367, 2010.
46. Tfelt-Hansen J, and Brown E. The calcium-sensing receptor in normal physiology and pathophysiology: a review. Crit Rev ClM Lab Sci 42: 35-70, 2005.
47. Vidyasagar S, Barmeyer C, Geibel J, Binder H J, and Rajendran V M. Role of short-chain fatty acids in colonic HCO(3) secretion. The American journal of physiology 288: G1217-1226, 2005.
48. Vidyasagar S, Rajendran V M, and Binder H J. Three distinct mechanisms of HCO₃-secretion in rat distal colon. The American journal of physiology 287: C612-621, 2004.
49. Wang Y, Chandra R, Samsa L, Gooch B, Fee B, Cook J M, Vigna S, Grant A, and Liddle R. Amino acids stimulate cholecystokinin release through the Ca2+sensing receptor. American journal of physiology: Gastrointestinal and liver physiology 300: G528-537, 2011.
50. Welsh M J, Smith P L, Fromm M, and Frizzell R A. Crypts are the site of intestinal fluid and electrolyte secretion. Science 218: 1219-1221, 1982.
51. Xiao F, Julie M, Li J, Riederer B, Yeruva S, Singh A K, Zheng L, Glage S, Kollias G, Dudeja P, Tian D A, Xu G, Zhu J, Bachmann O, and Seidler U. Loss of downregulated in adenoma (DRA) impairs mucosal HCO3(−) secretion in murine ileocolonic inflammation. Inflammatory bowel diseases 18: 101-111, 2012.
52. Xiao F, Yu Q, Li J, Johansson M E, Singh A K, Xia W, Riederer B, Engelhardt R, Montrose M, Soleimani M, Tian D A, Xu G, Hansson G C, and Seidler U. Slc26a3 deficiency is associated with loss of colonic HCO3 secretion, absence of a firm mucus layer and barrier impairment in mice. Acta physiologica 211: 161-175, 2013.

We claim:

1. A method of treating cystic fibrosis (CF) in a subject, the method comprising administering, to a subject who has been diagnosed with CF, a composition comprising a CaSR agonist.

2. The method of claim 1, wherein the composition comprises an extracellular calcium-sensing receptor (CaSR) orthosteric agonist.

3. The method of claim 1, wherein the composition comprises an agent capable of stimulating colonic bicarbonate (HCO₃⁻) secretion in a cystic fibrosis transmembrane conductance regulator (CFTR)-independent manner.

4. The method of claim 3, wherein the composition comprises a CaSR orthosteric agonist and the agent capable of stimulating colonic HCO₃⁻ secretion in a CFTR-independent manner.

5. The method of claim 4, wherein the method comprises administering R568, R467, Calindol, Cinacalcet, L-phenylalanine, L-tryptophan, L-tyrosine, or L-histidine.

6. The method of claim 2, wherein the CaSR orthosteric agonist is Ca²⁺, Mg²⁺, Al³⁺, Sr²⁺, Mn²⁺, Ni²⁺, Gd³⁺, Ba²⁺, neomycin, gentamycin, tobramycin, spermine, spermidine, or putrescine.

7. The method of claim 3, wherein the agent capable of stimulating colonic HCO₃⁻ secretion in a CFTR-independent manner is Cl⁻ or SCFA.

8. The method of claim 1, comprising administering the composition to the subject via inhalation or enema.

9. The method of claim 8, wherein administering the composition to the subject via inhalation is performed by a device suitable for administration of the composition to the subject via inhalation.

10. The method of claim 9, wherein the device is an aerosol delivery device.

11. The method of claim 10, wherein the device is an inhaler, atomizer, nebulizer, vaporizer, insufflator or puffer.

12. A composition for treatment of CF, the composition comprising a calcimimetic.

13. The composition of claim 12, further comprising a CaSR orthosteric agonist.

14. The composition of claim 12, further comprising an agent capable of stimulating colonic HCO₃⁻ secretion in a CFTR-independent manner.

15. The composition of claim 12, further comprising an CaSR orthosteric agonist and an agent capable of stimulating colonic HCO₃⁻ secretion in a CFTR-independent manner.

16. The composition of claim 12, wherein the calcimimetic is R568, 8467, Calindol, Cinacalcet, L-phenylalanine, L-tryptophan, L-tyrosine, or L-histidine.

17. The composition of claim 13, wherein the CaSR orthosteric agonist is Ca²⁺, Mg²⁺, Al³⁺, Sr²⁺, Mn²⁺, Ni²⁺, Gd³⁺, Ba²⁺, neomycin, gentamycin, tobramycin, spermine, spermidine, or putrescine.

18. The composition of claim 14, wherein the agent capable of stimulating colonic HCO₃⁻ secretion in a CFTR-independent manner is Cl⁻ or SCFA.

19. The composition of claim 12, formulated for administration to a subject via inhalation.

20. A device comprising the composition of claim 12, wherein the device is suitable for administering the composition to a subject via inhalation.

21. The device of claim 20, wherein the device is an inhaler, an atomizer, a nebulizer, a vaporizer, an insufflator or a puffer.
